(19) United States
(12) Patent Application Publication
(10) Pub. No.: US 2005/0203346 A1
(43) Pub. Date: Sep. 15, 2005

(54) Wound retractor device

(76) Inventors: Frank Bonadio, Bray (IE); Trevor Vaugh, Birr (IE); John Butler, Blackrock (IE); Catherine Deegan, Clontarf (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/086,660

(22) Filed: Mar. 23, 2005

Related U.S. Application Data
(63) Continuation-in-part of application No. 10/374,523, filed on Feb. 27, 2003.
Application No. 10/374,523 is a continuation of application No. 09/849,341, filed on May 7, 2001, now Pat. No. 6,582,364.
Application No. 09/849,341 is a continuation of application No. 09/688,138, filed on Oct. 16, 2000, now Pat. No. 6,254,534.
(60) Provisional application No. 60/555,400, filed on Mar. 23, 2004.

(30) Foreign Application Priority Data
Feb. 18, 2000 (EP) 00650010.2
Oct. 14, 1999 (IE) 990861
Dec. 16, 1999 (IE) 991053

(51) Int. Cl. A61B 1/32

(52) U.S. Cl. 600/208

(57) ABSTRACT

A wound retractor device (1) comprises a distal ring member (4) for insertion into a wound opening (3), a proximal ring member (2) for location externally of the wound opening (3), and a cylindrical connecting sleeve (5), which extends between the distal ring member (4) and the proximal ring member (2), to retract laterally the sides of a wound opening (3). The device (1) further comprises a rigid engagement ring member (11) for facilitating engagement of the device (1), and manipulation of the device (1) into a desired location and/or orientation. In use, the distal ring member (4) is inserted into the wound opening (3), and the proximal ring member (2) is located externally of the wound opening (3), with the connecting sleeve (5) extending between the proximal ring member (2) and the distal ring member (4). The connecting sleeve (5) is then pulled proximally to retract laterally the sides of the wound opening (3). The engagement ring member (11) may then be engaged, for example by means of a surgeon gripping the engagement ring member (11), and the device (1) manipulated into a desired location and/or orientation.

This is a continuation-in-part of application Ser. No. 10/374,523, filed Feb. 27, 2003, which is a continuation of application Ser. No. 09/849,341, filed May 7, 2001, now U.S. Pat. No. 6,582,364, which is a continuation of application Ser. No. 09/688,138, filed Oct. 16, 2000, now U.S. Pat. No. 6,254,534 B1. This application claims the benefit of U.S. Provisional Application No. 60/555,400, filed on Mar. 23, 2004. The contents of all of the above-listed applications are incorporated herein by reference.

### INTRODUCTION

This invention relates to a wound retractor device, and to a method for retracting a wound opening.

### STATEMENTS OF INVENTION

According to the invention there is provided a wound retractor device comprising:
a distal member for insertion into a wound opening;
a proximal member for location externally of the wound opening;
a connecting member extending between the distal member and the proximal member to retract laterally the sides of the wound opening; and
an engagable member for facilitating engagement of the wound retractor device and manipulation of the wound retractor device into a desired location and/or orientation.

In one embodiment the proximal member comprises the engagable member.

In another embodiment the engagable member comprises a separate engagement member for location externally of a wound opening. The engagement member may be mountable to the proximal member. In one case the engagement member is mountable to the proximal member in a snap-fit manner. The engagement member may comprise a female recess, into which the proximal member is receivable to mount the engagement member to the proximal member. In one case the female recess is substantially “C”-shaped in cross-section. An opening of the female recess may face radially inwardly. An opening of the female recess may face radially outwardly. An opening of the female recess may face distally.

In one case the engagement member comprises a lip for engagement with the proximal member to mount the engagement member to the proximal member. The lip may protrude radially outwardly. The engagement member may be substantially “L”-shaped in cross-section.

In one embodiment the proximal member comprises a ring. In another embodiment the engagement member comprises a ring.

The engagement member may comprise a grippable element for facilitating gripping of the engagement member. In one case the grippable element is selectively movable relative to a main body portion of the engagement member. The grippable element may be slidable relative to the main body portion. The grippable element may be mountable to the main body portion. In one case the grippable element is mountable to the main body portion in a snap-fit manner. In one case the grippable element comprises a female recess, into which the main body portion is receivable to mount the grippable element to the main body portion. The female recess may be substantially “C”-shaped in cross-section.

In another embodiment the main body portion comprises a channel and the grippable element is movably received in the channel. The channel may comprise a slot in the main body portion. The channel may extend around part of the circumference of the main body portion.

In one embodiment the grippable element comprises at least one hook formation.

The grippable element may comprise a handle. In one case the handle extends radially outwardly from the engagement member.

The device may comprise a support to support the engagable member in a manipulated location and/or orientation. In one case at least part of the support is configured for positioning substantially above a wound opening to support the engagable member from above. The support may comprise a tensioner to exert a tensile force on the engagable member. In one case the magnitude and/or direction of the tensile force is adjustable. The tensioner may comprise one or more wires.

In one embodiment the support comprises one or more arm members for extending from one or more first locations spaced a substantial distance from a wound opening to one or more second locations adjacent the wound opening. The location and/or orientation of the arm member may be adjustable. The support may comprise one or more leg members for supporting the one or more arm members in desired locations and/or orientations. In one case the support comprises a base member for supporting the one or more leg members. The base member may be configured for location resting on a treatment bed. In one case the base member is configured to at least partially extend beneath the body of a patient being treated.

In another aspect, the invention provides a method for retracting a wound opening, the method comprising the steps of:
providing a wound retractor device comprising a distal member, a proximal member and a connecting member extending between the distal member and the proximal member;
inserting the distal member into the wound opening, and locating the proximal member externally of the wound opening, with the connecting member extending between the distal member and the proximal member to retract laterally the sides of the wound opening; and
engaging the wound retractor device, and manipulating the wound retractor device into a desired location and/or orientation.

In one embodiment the proximal member is engaged to manipulate the wound retractor device into a desired location and/or orientation.

In another embodiment the wound retractor device comprises a separate engagement member located externally of the wound opening, and the engagement member is engaged to manipulate the wound retractor device into a desired location and/or orientation. In one case the method comprises the step of mounting the engagement member to the proximal member. The method may comprise the step of gripping the engagement member at a first region of the engagement member. In one case the method comprises the step of moving the region of gripping of the engagement member from the first region to a second region of the engagement member. The method may comprise the step of sliding the region of gripping from the first region to the second region.

In a further case the method comprises the step of supporting the wound retractor device in a manipulated position and/or orientation.

### BRIEF DESCRIPTION OF THE DRAWINGS

The invention will be more clearly understood from the following description of some embodiments thereof, given by way of example only, with reference to the accompanying drawings, in which:—

FIG. 1 is a cross-sectional, side view of a wound retractor device according to the invention;

FIG. 2 is a cross-sectional, side view of the device of FIG. 1, in use;

FIGS. 3 to 8 are partially cut-away, perspective views of the device of FIG. 1, in use;

FIG. 9 is a perspective view of a part of another wound retractor device according to the invention;

FIG. 10 is a cut-away, perspective view of the part of FIG. 9;

FIGS. 11 and 12 are cross-sectional, side views of the device of FIG. 9, in use;

FIG. 13 is a perspective view of a part of another wound retractor device according to the invention;

FIG. 14 is a cut-away, perspective view of the part of FIG. 13;

FIG. 15 is a cross-sectional, side view of the device of FIG. 13, in use;

FIG. 16 is a perspective view of a part of another wound retractor device according to the invention;

FIG. 17 is a cross-sectional, side view of the device if FIG. 16, in use;

FIGS. 18 and 19 are cross-sectional, side views of a further wound retractor device according to the invention, in use;

FIG. 20 is a cut-away, perspective view of the device of FIGS. 18 and 19, in use;

FIGS. 21 and 22 are plan views of another wound retractor device according to the invention;

FIG. 23 is a cut-away, perspective view of the device of FIGS. 21 and 22, in use;

FIGS. 24 and 25 are cross-sectional, side views of the device of FIGS. 21 and 22, in use;

FIGS. 26 and 27 are cross-sectional, side views of another wound retractor device according to the invention, in use;

FIGS. 27(a) and 27(b) are cross-sectional, side views of another wound retractor device according to the invention, in use;

FIGS. 27(c) and 27(d) are cross sectional, side views of a further wound retractor device according to the invention, in use;

FIGS. 28 and 29 are plan views of another wound retractor device according to the invention, in use;

FIG. 30 is an end view of the device of FIGS. 28 and 29, in use;

FIG. 31 is a side view of the device of FIGS. 28 and 29, in use;

FIG. 32 is another plan view of the device of FIGS. 28 and 29, in use;

FIG. 33 is another side view of the device of FIGS. 28 and 29, in use;

FIG. 34 is a plan view of another wound retractor device according to the invention, in use;

FIG. 35 is an end view of the device of FIG. 34, in use;

FIG. 36 is a side view of another wound retractor device according to the invention, in use;

FIG. 37 is a side view of a further wound retractor device according to the invention, in use;

FIG. 38 is a plan view of another wound retractor device according to the invention, in use;

FIG. 39 is a side view of the device of FIG. 38, in use;

FIGS. 40 and 41 are end views of the device of FIG. 38, in use;

FIG. 42 is a side view of another wound retractor device according to the invention, in use;

FIG. 43 is a plan view of the device of FIG. 42, in use;

FIG. 44 is an end view of the device of FIG. 42, in use;

FIGS. 45 and 46 are plan views of a further wound retractor device according to the invention, in use; and

FIG. 47 is a side view of the device of FIGS. 45 and 46, in use.

### DETAILED DESCRIPTION

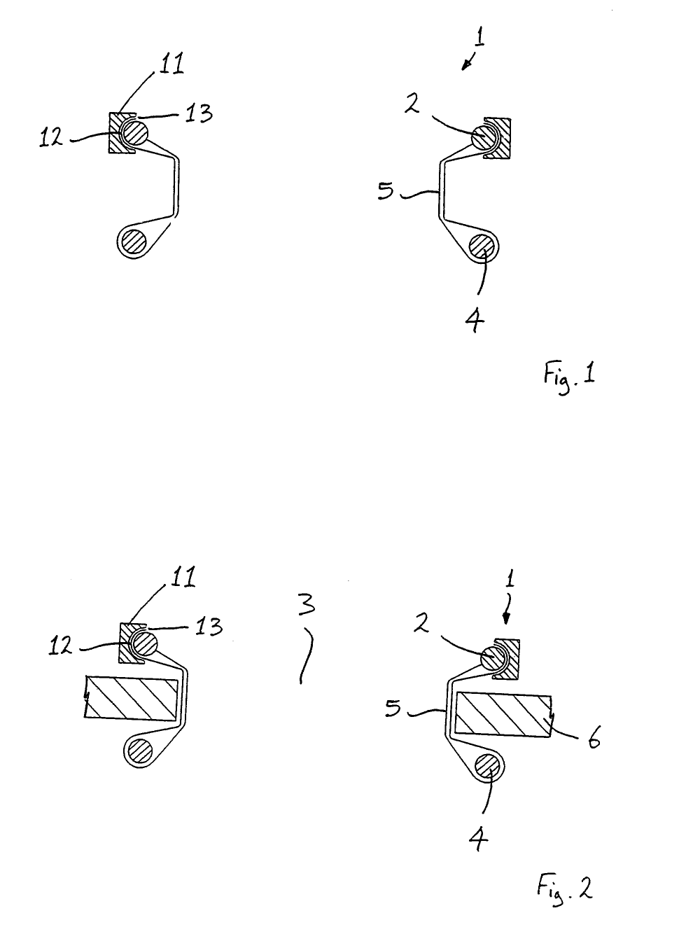

Referring to the drawings, and initially to FIGS. 1 and 2 thereof, there is illustrated a wound retractor device 1 according to the invention.

The device 1 comprises a distal ring member 4 for insertion into a wound opening 3, a proximal ring member 2 for location externally of the wound opening 3, and a connecting member. In this case, the connecting member is provided in the form of a cylindrical connecting sleeve 5, which extends between the distal ring member 4 and the proximal ring member 2, to retract laterally the sides of a wound opening 3, as illustrated in FIG. 2.

A first end of the sleeve 5 is fixedly attached to the proximal ring member 2, and the sleeve 5 extends distally from the proximal ring member 2 in a first layer to the distal ring member 4. The sleeve 5 is looped around the distal ring member 4 and extends proximally to the proximal ring member 2 in a second layer. As illustrated in FIG. 2, the second layer is located radially outwardly of the first layer. A second end of the sleeve 5 remains detached from the proximal ring member 2. This arrangement of the wound retractor device 1 results in a self-locking retractor. This arrangement is described in further detail in International patent application No. PCT/IE2003/000141, the relevant contents of which are incorporated herein by reference.

The device 1 further comprises an engagable member for facilitating engagement of the device 1, and manipulation of the device 1 into a desired location and/or orientation.

In this case, the engagable member is provided by a rigid engagement ring member 11, suitable for location, in use, externally of the wound opening 3. The engagement ring member 11 is substantially “C”-shaped in cross-section, and comprises a female recess 12 into which the proximal ring member 2 is receivable to mount the engagement ring member 11, in a snap-fit manner to the proximal ring member 2. As illustrated in FIGS. 1 and 2, the opening 13 of the female recess 12 faces radially inwardly.

In use, the distal ring member 4 is inserted into the wound opening 3, and the proximal ring member 2 is located externally of the wound opening 3, with the connecting sleeve 5 extending between the proximal member 2 and the distal ring member 4. The connecting sleeve 5 is then pulled proximally to retract laterally the sides of the wound opening 3 (FIG. 3). The method of retracting the sides of the wound opening 3 is described in further detail in International patent application No. PCT/IE2003/000141, the relevant contents of which are incorporated herein by reference. The engagement ring member 11 may then be engaged, for example by means of a surgeon gripping the engagement ring member 11, and the device 1 manipulated into a desired location and/or orientation.

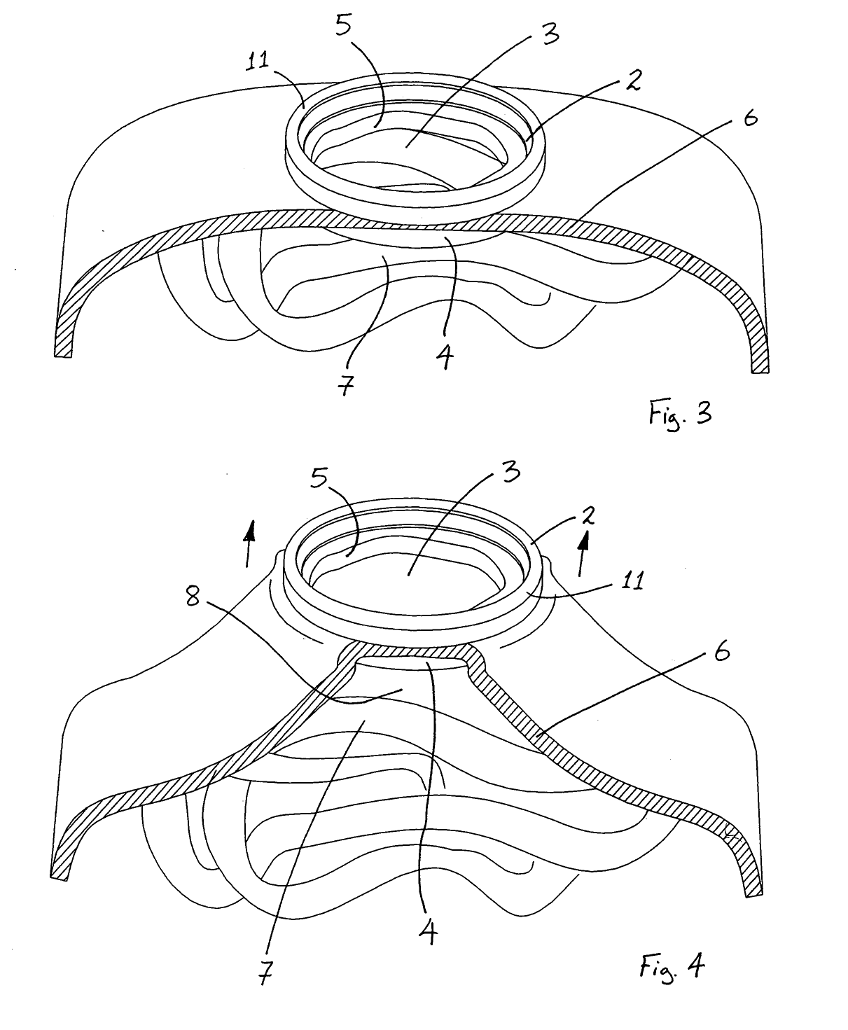

For example, both sides of the engagement ring member 11 may be pulled vertically upwards, as illustrated in FIG. 4, to create additional working space 8 between the peritoneum 6 and the internal organs 7. When the device 1 is retracting the wound opening 3 in the at-rest position (FIG. 3), the internal organs 7 are packed closely together against the peritoneum 6. The additional working space 8 in the manipulated position (FIG. 4) enables a surgeon to carry out a surgical procedure through the wound opening 3 without being hindered by the presence of the surrounding internal organs 7. In this manner, the lifting of the peritoneum 6 provides the surgeon with enhanced access to the wound interior.

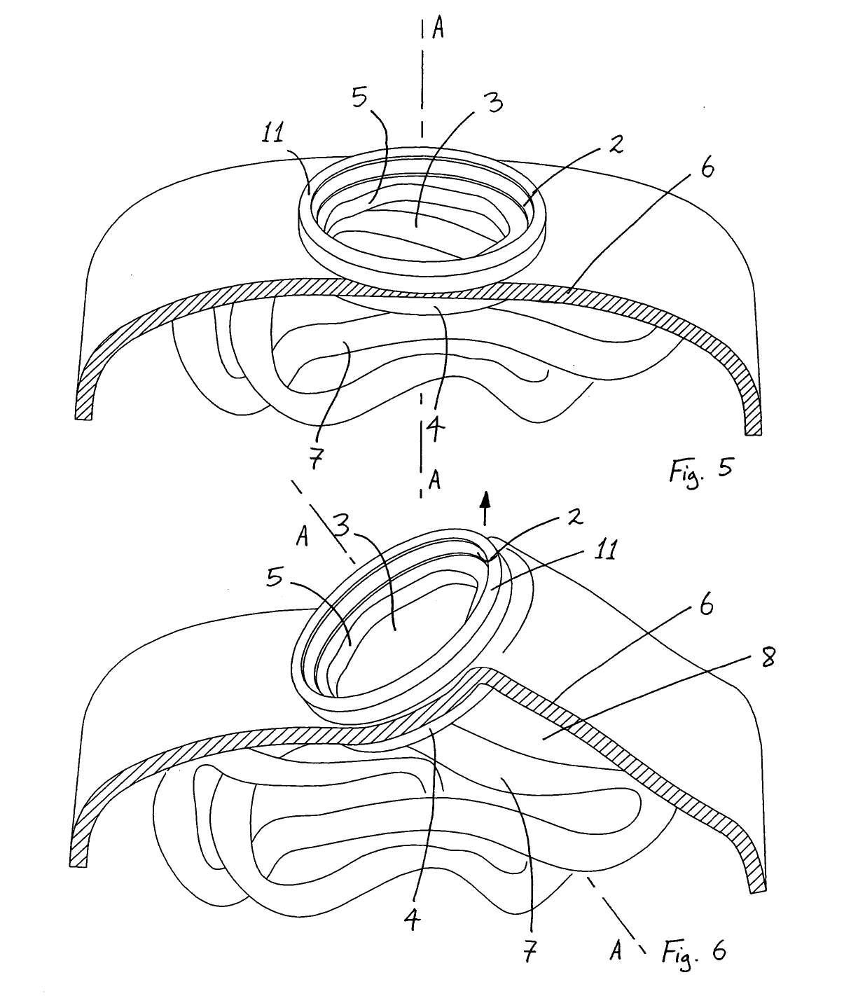

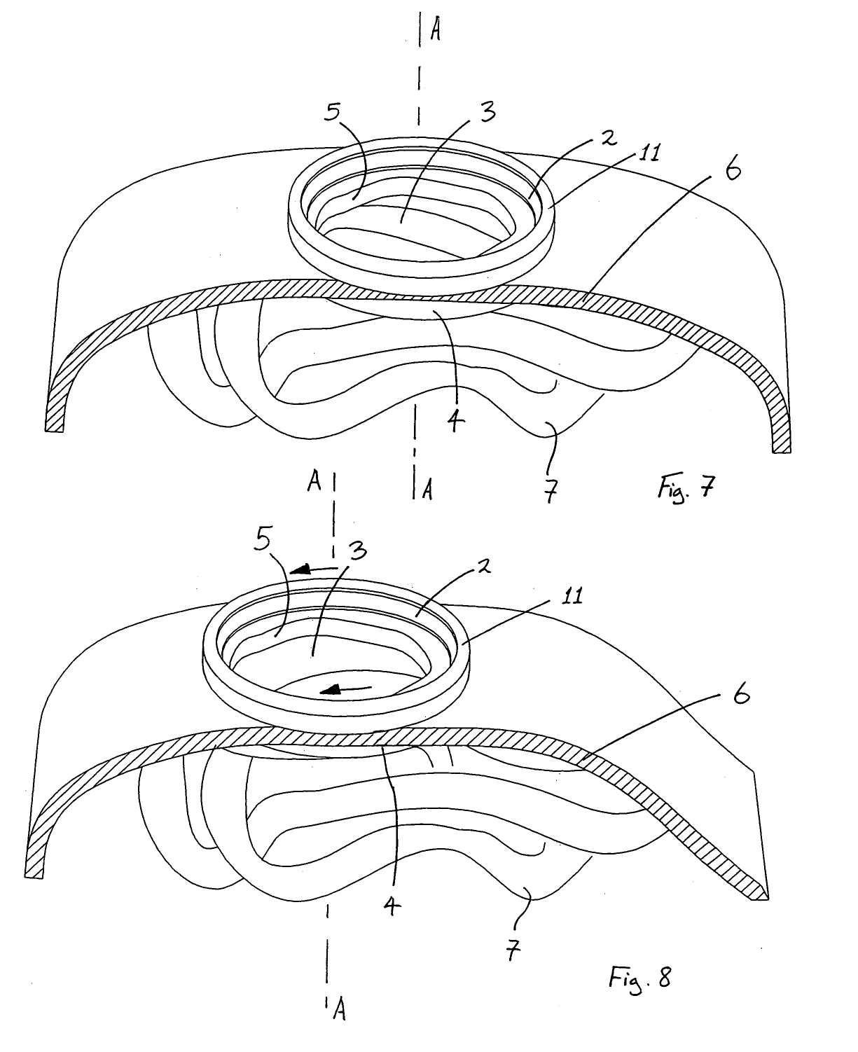

Alternatively only one side of the engagement ring member 11 may be pulled vertically upwards, as illustrated in FIG. 6, to create an alternative angle of vision through the wound opening 3, and also to create additional working space 8 between the peritoneum 6 and the internal organs 7. In this manner, the surgeon may easily view the portion of the internal organs 7 which are visible along the longitudinal axis A-A of the device 1 when the device 1 is retracting the wound opening 3 in the at-rest position (FIG. 5), and also the portion of the internal organs 7 which are visible along the longitudinal axis A-A of the device 1 when the device 1 is retracting the wound opening 3 in the manipulated position (FIG. 6). In particular, additional artificial vision means, such as a scope, are not required. Thus lifting the peritoneum 6 provides the surgeon with enhanced vision of the wound interior.

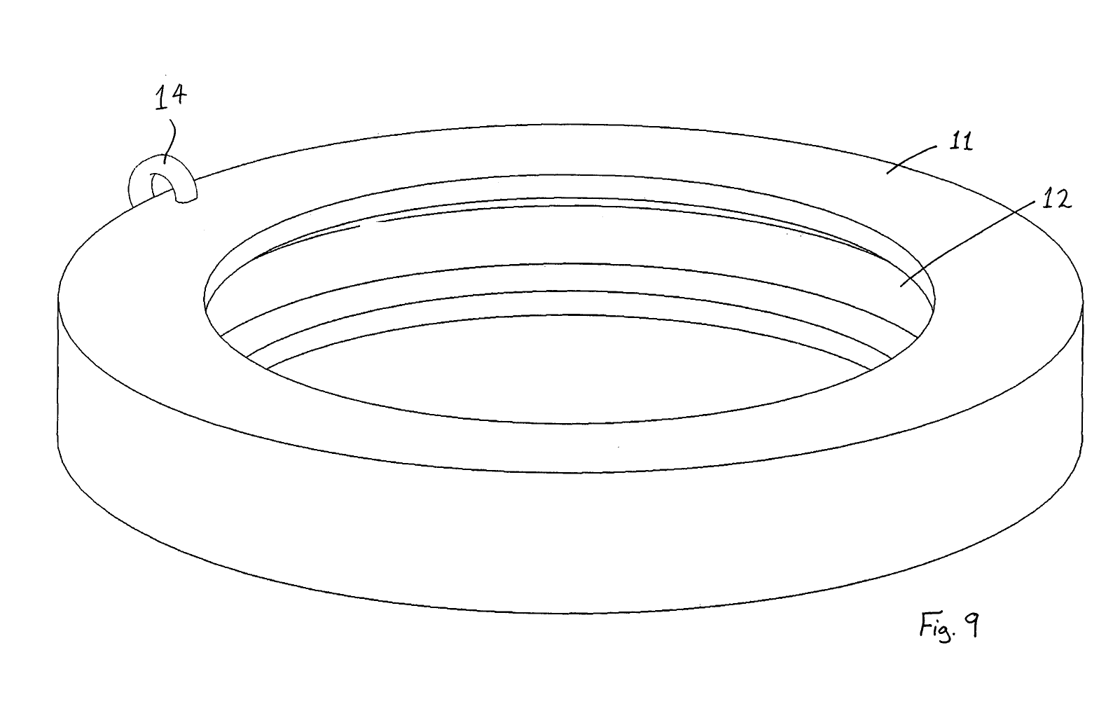

As a further alternative, both sides of the engagement ring member 11 may be pulled horizontally across, as illustrated in FIG. 8, to create an alternative angle of vision through the wound opening 3. In this manner the surgeon may easily view the portion of the internal organs 7 which are visible along the longitudinal axis A-A of the device 1 when the device 1 is retracting the wound opening 3 in the at-rest position (FIG. 7), and also the portion of the internal organs 7 which are visible along the longitudinal axis A-A of the device 1 when the device 1 is retracting the wound opening 3 in the manipulated position (FIG. 8). In particular, additional artificial vision means, such as a scope, are not required.

Manipulating the device 1 thus provides the surgeon with access to or vision of a portion of the internal organs 7 at a variety of locations within the wound interior.

It will be appreciated that the device 1 may be manipulated into a variety of different locations and/or orientations, in addition to or as alternatives to the manipulations described previously with reference to FIGS. 3 to 8.

It will be appreciated that the engagable member may alternatively be provided directly by the proximal ring member 2.

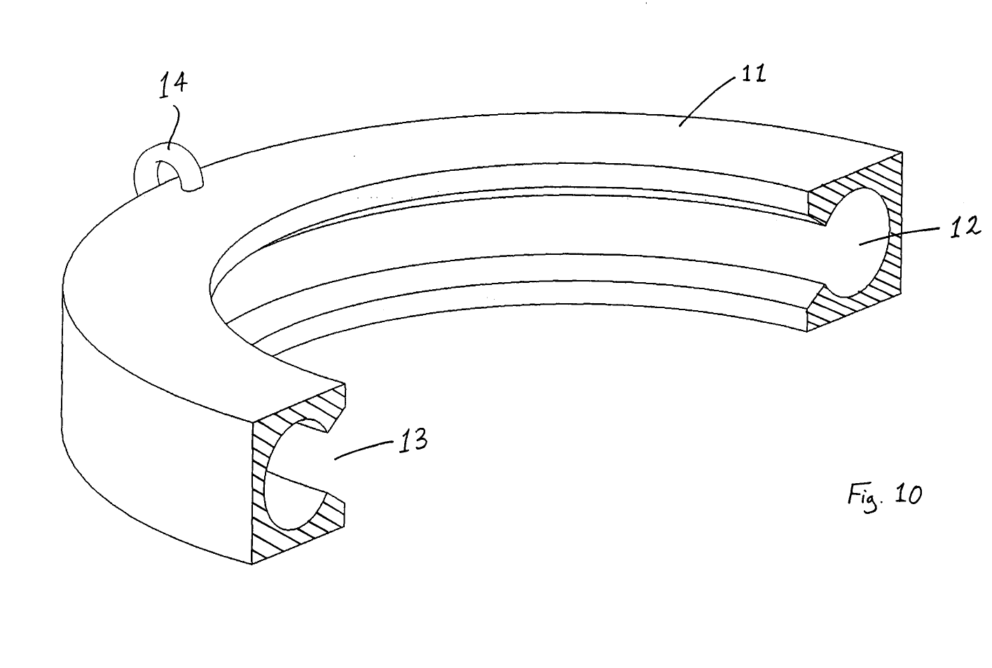

Referring to FIG. 9 to 12 there is illustrated another wound retractor device 10 according to the invention.

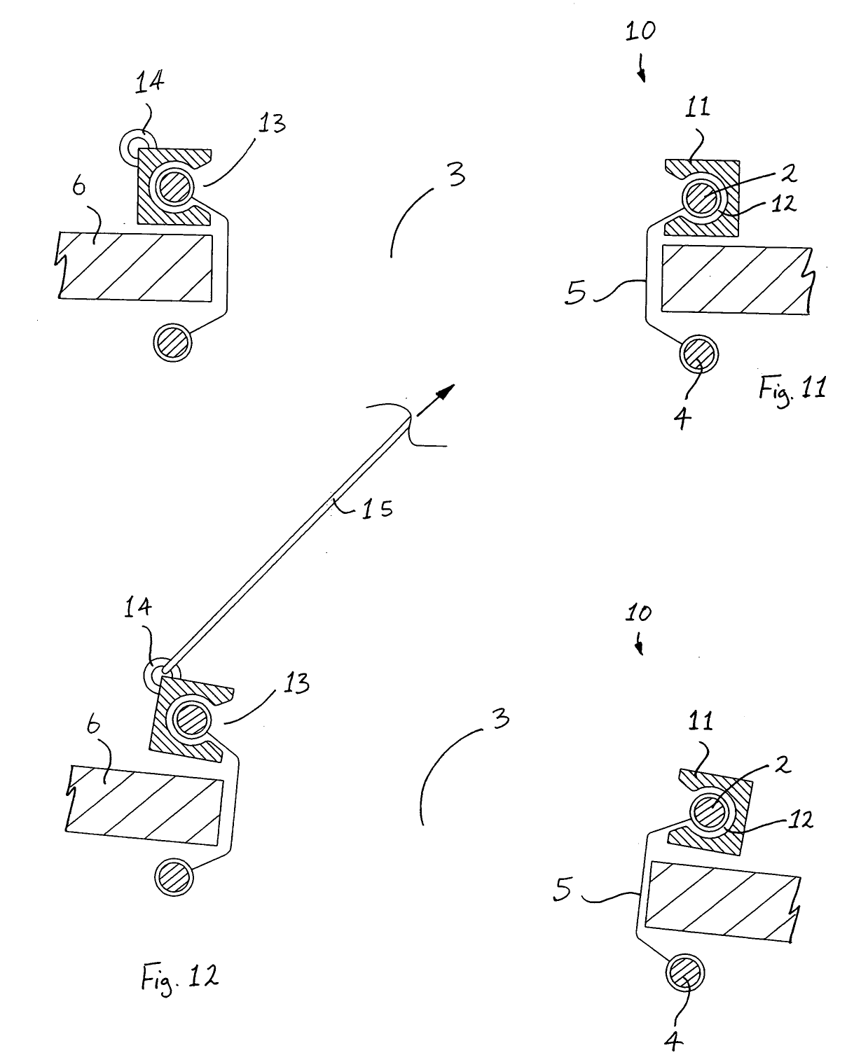

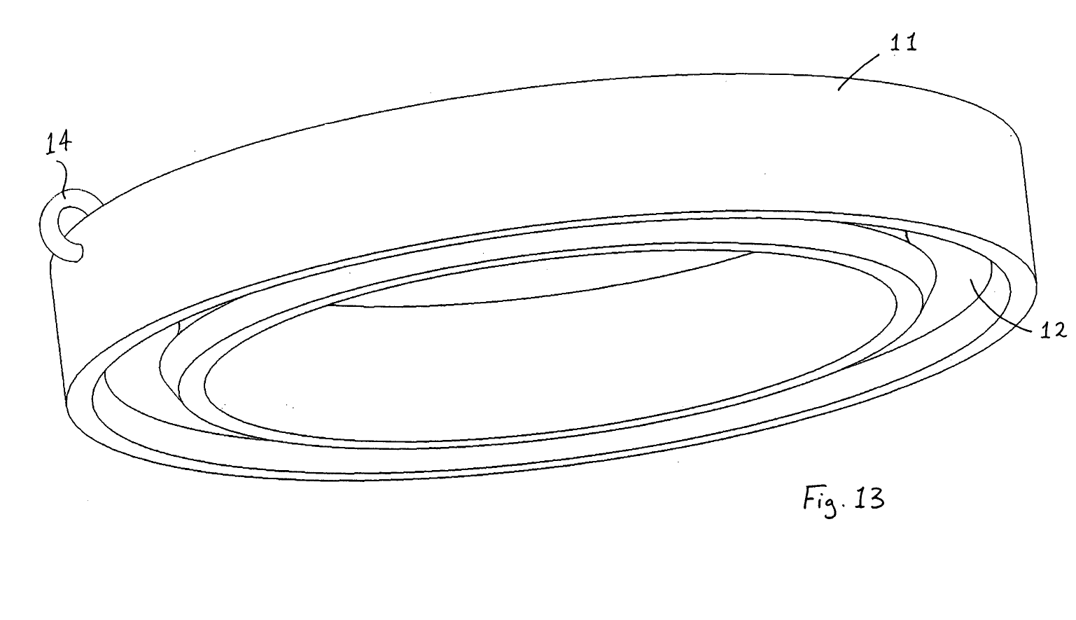

The engagement ring member 11 comprises a hook formation 14 which provides a means of gripping the engagement ring member 11. For example, a wire or cable 15 may be looped through the hook formation 14 to enable the engagement ring member 11 to be gripped, and thus to enable the device 10 to be manipulated into a desired position and/or orientation, as illustrated in FIG. 12. In this case, the hook formation 14 is fixedly attached to the engagement ring member 11.

It will be appreciated that the opening 13 of the female recess 12 could alternatively face radially outwardly.

In use, the distal ring member 4 is inserted into the wound opening 3, and the proximal ring member 2 is located externally of the wound opening 3, with the connecting sleeve 5 extending between the distal ring member 4 and the proximal ring member 2 to retract laterally the sides of the wound opening 3. The engagement ring member 11 is then snap-fit mounted to the proximal ring member 2. The engagement ring member 11 may be gripped, and the device 10 manipulated into a desired location and/or orientation.

The engagement ring member 11 may be gripped by means of the hook formation 14, for example by looping the wire 15 through the hook formation 14 and exerting a tensile force on the wire 15. Alternatively the engagement ring member 11 may be gripped directly by the surgeon's hand.

The rigid engagement ring member 11 is particularly suitable for use with a floppy proximal ring member 2. Using a floppy proximal ring member 2, it may be easier to insert the distal ring member 4 in to the wound opening 3. For example if scrunching up of the distal ring member 4 is necessary, this may be easier to achieve when the proximal ring member 2 is floppy.

In this case the sleeve 5 extends between the distal ring member 4 and the proximal ring member 2 in a single layer. The engagable member aspect of the invention is suitable for use with a variety of different configurations for the distal ring/proximal ring/connecting member.

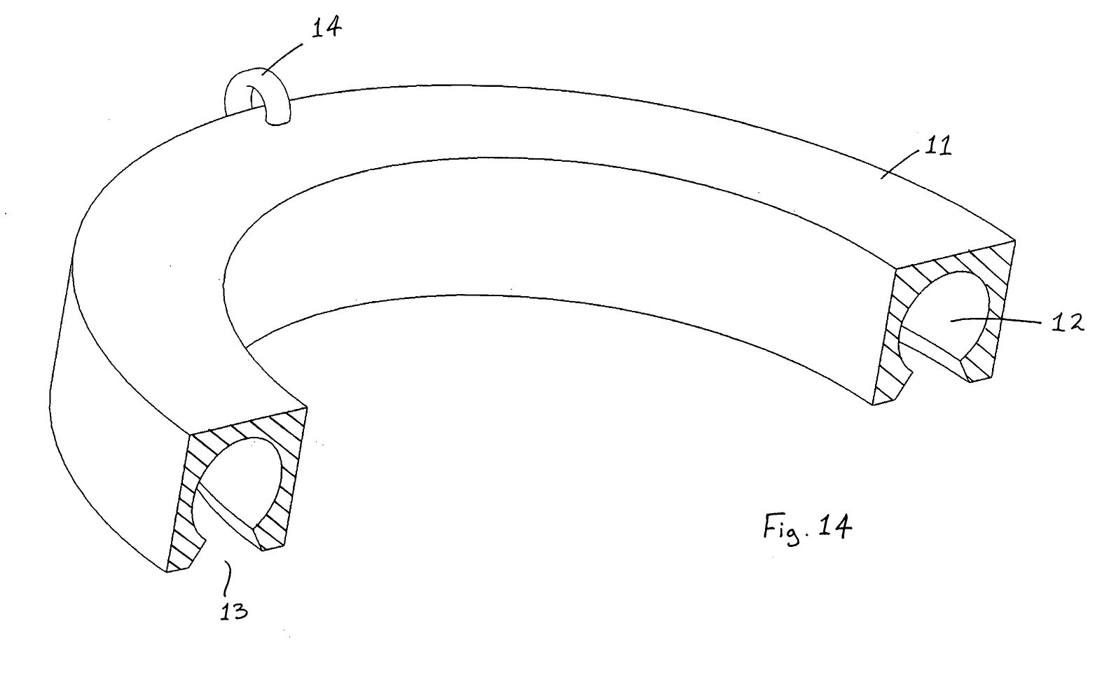

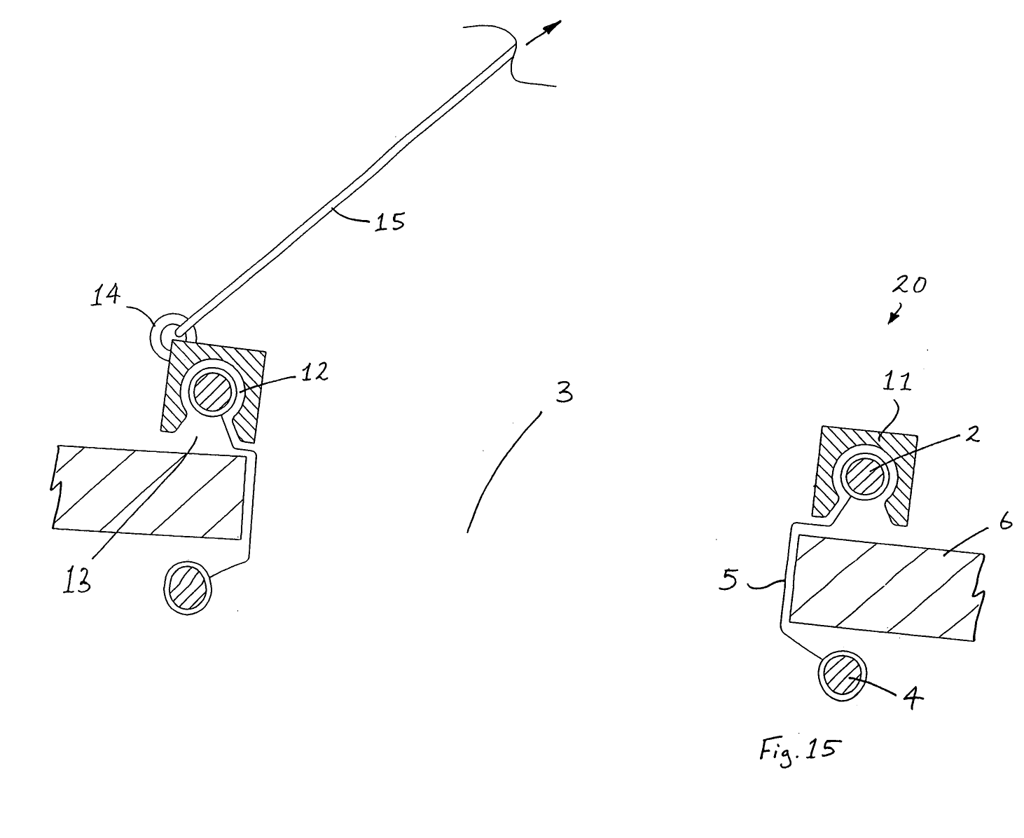

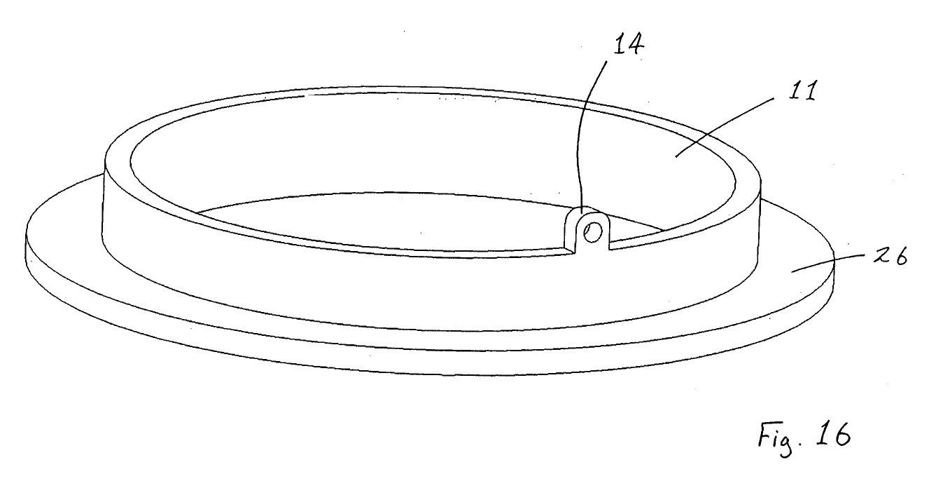

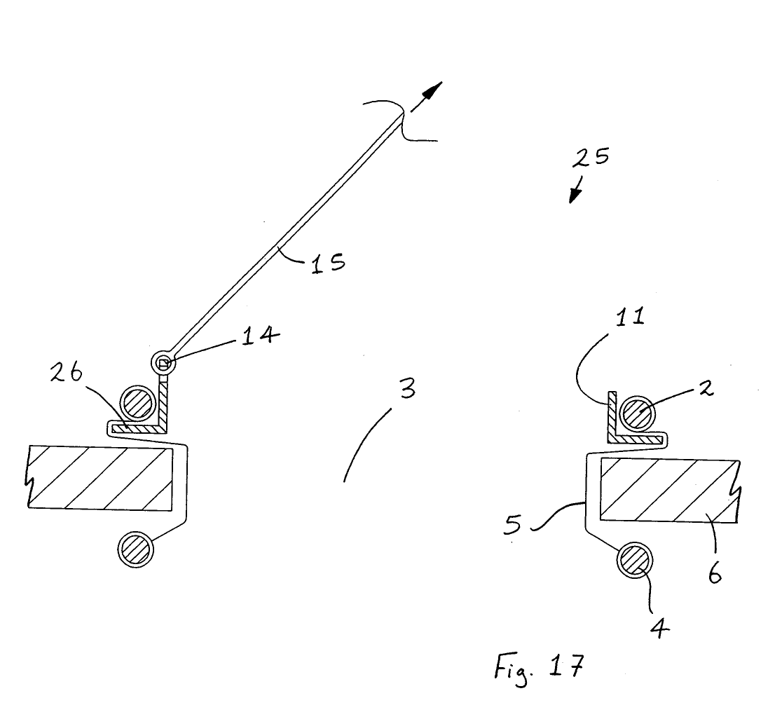

In FIGS. 13 and 15, there is illustrated a further wound retractor device 20 according to the invention, which is similar to the device 10 of FIGS. 9 to 12, and similar elements in FIGS. 13 to 15 are assigned the same references numerals.

In this case the opening 13 of the female recess 12 faces distally, downwardly towards the peritoneum 6.

FIGS. 16 and 17 illustrate another wound retractor device 25 according to the invention, which is similar to the device 10 of FIGS. 9 to 12, and similar elements in FIGS. 16 and 17 are assigned the same reference numerals.

In this case, the rigid engagement ring member 11 is substantially “L” shaped in cross-section, and comprises a radially outwardly protruding lip 26 for engagement with the proximal ring member 2 to mount the engagement ring member 11 to the proximal ring member 2 (FIG. 17).

The L-shaped engagement ring member 11 of FIGS. 16 and 17 is particularly suitable for use with a floppy proximal ring member 2.

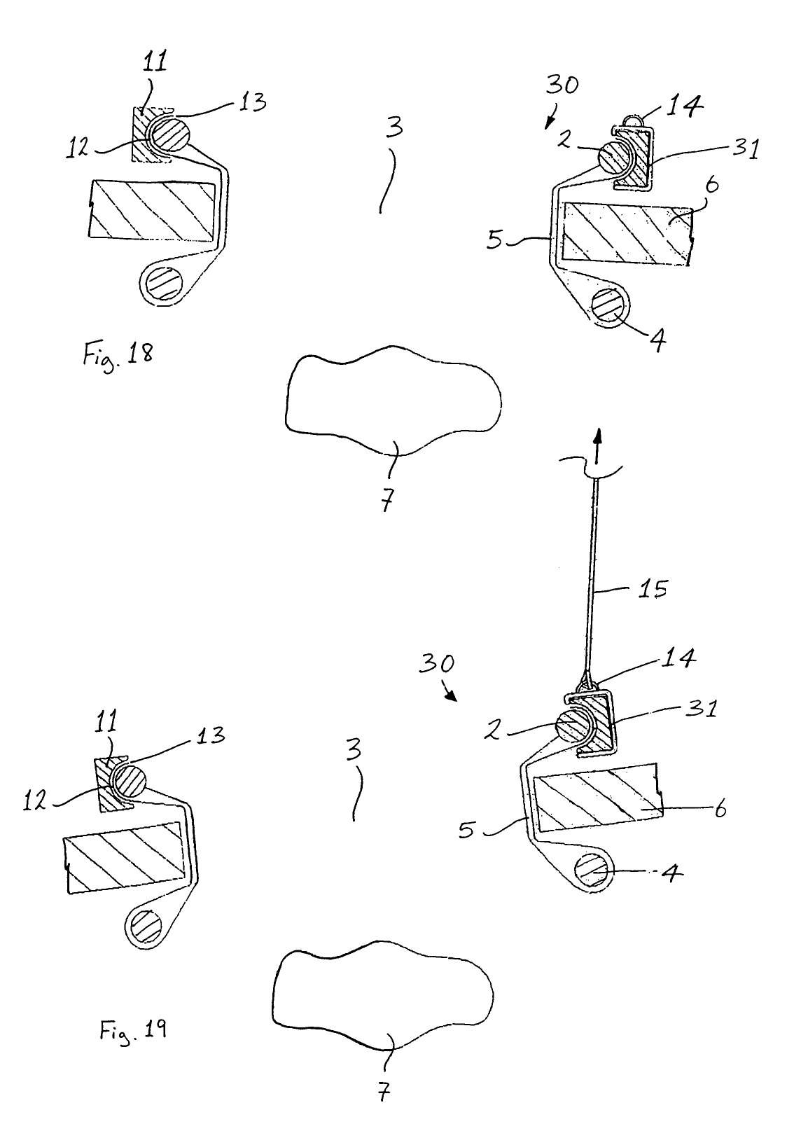

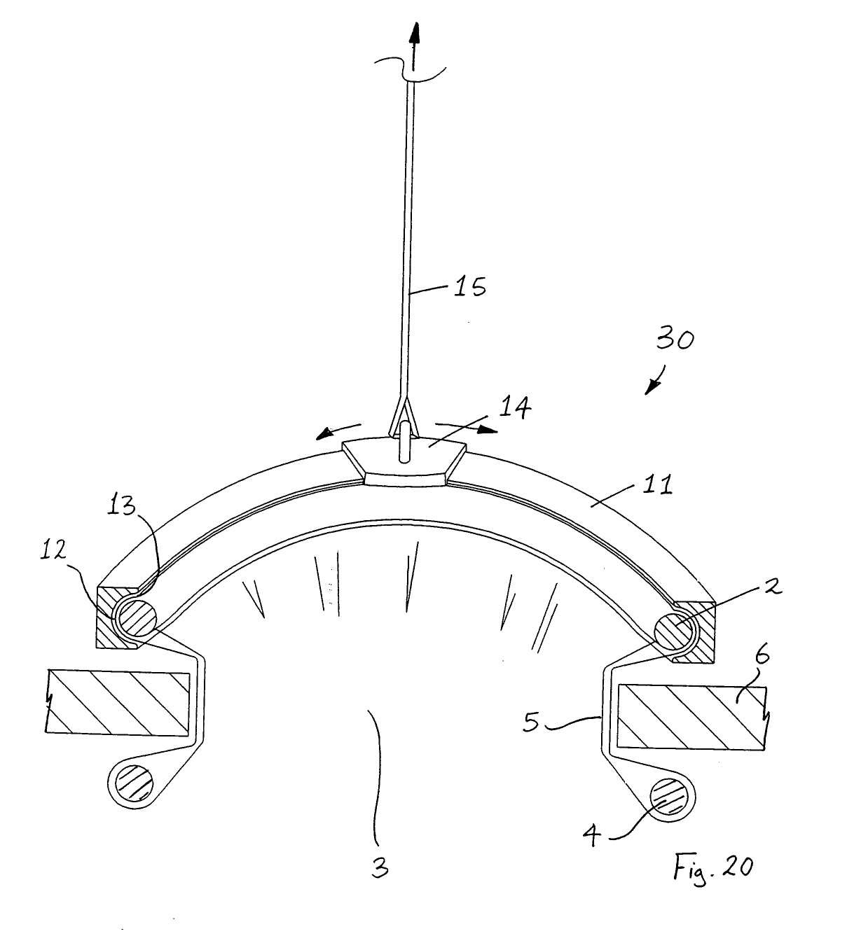

Referring now to FIGS. 18 to 20 there is illustrated another wound retractor device 30 according to the invention, which is similar to the device 1 of FIGS. 1 to 8, and similar elements in FIGS. 18 to 20 are assigned the same reference numerals.

In this case, the hook formation 14 is substantially “C”-shaped in cross-section, and comprises a female recess 31 into which the engagement ring member 11 is receivable to mount the hook formation 14 to the engagement ring member 11 in a snap-fit manner. The hook formation 14 is thus selectively slidably movable relative to the engagement ring member 11. In this manner, the region of the engagement ring member 11 which is being gripped may be easily slidably moved to achieve manipulation of the device 30 into a desired location and/or orientation. The portion of the engagement ring member 11 where the hook formation 14 is located will be pulled upwardly when the wire 15 is pulled upwardly.

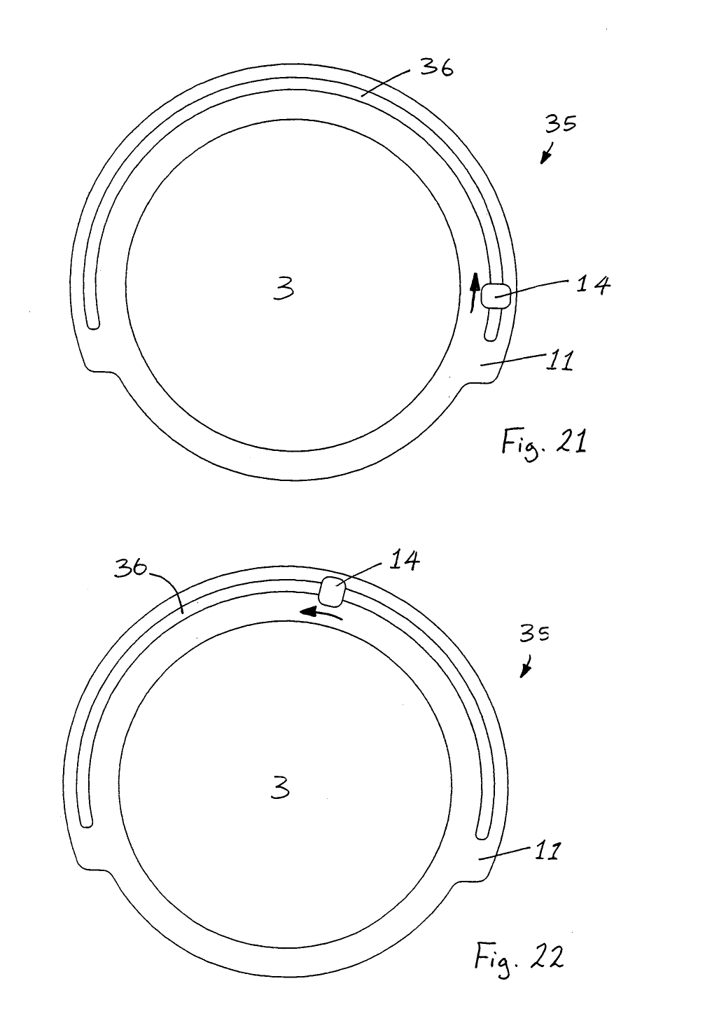

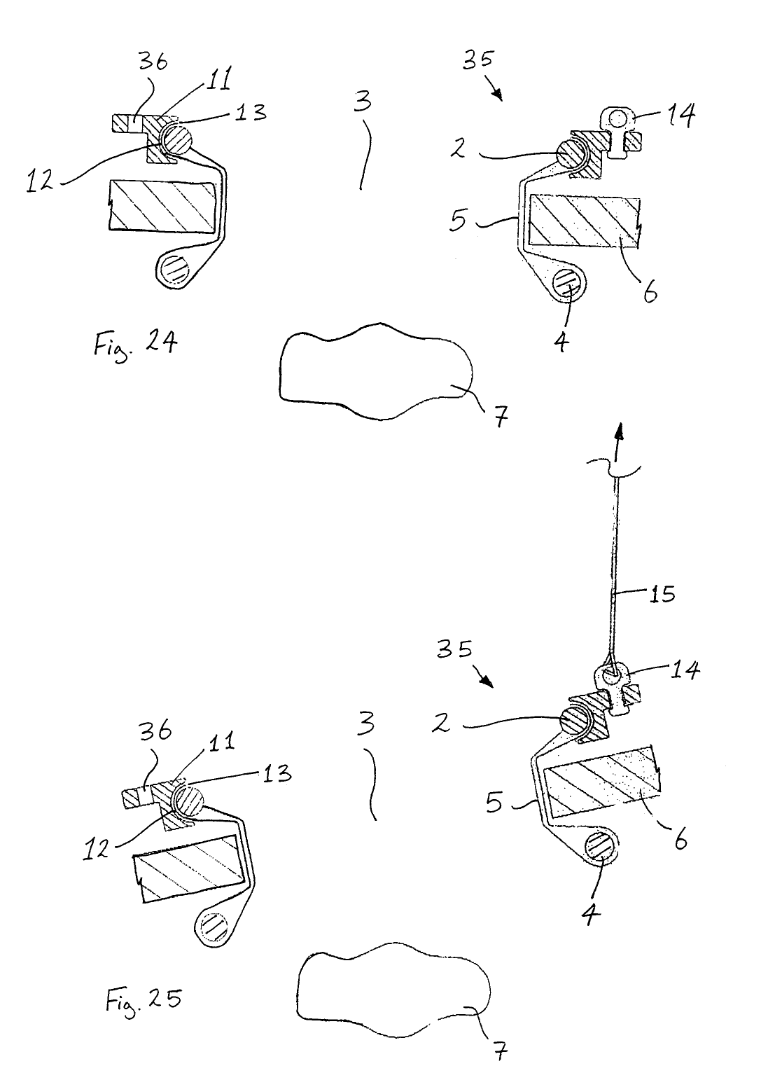

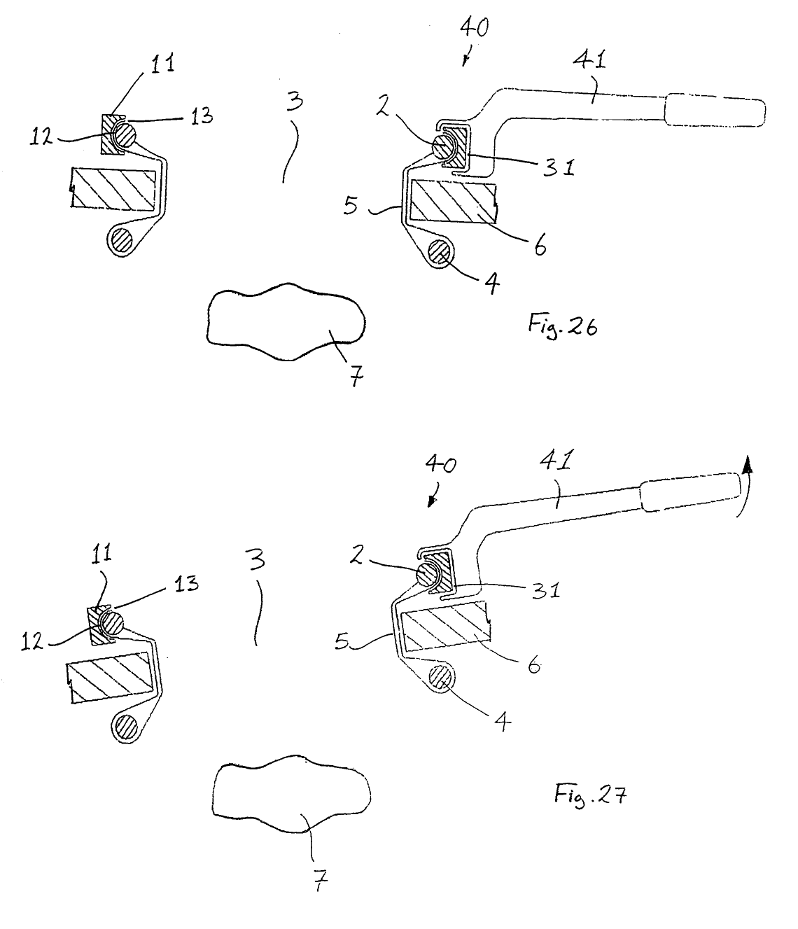

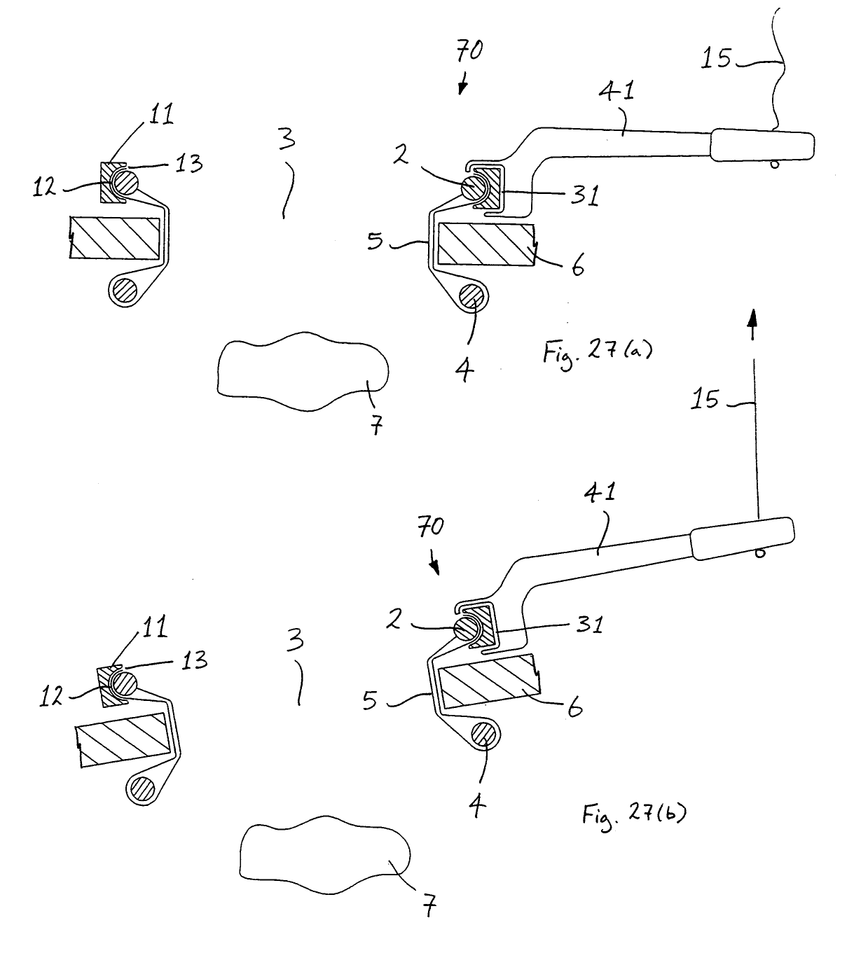

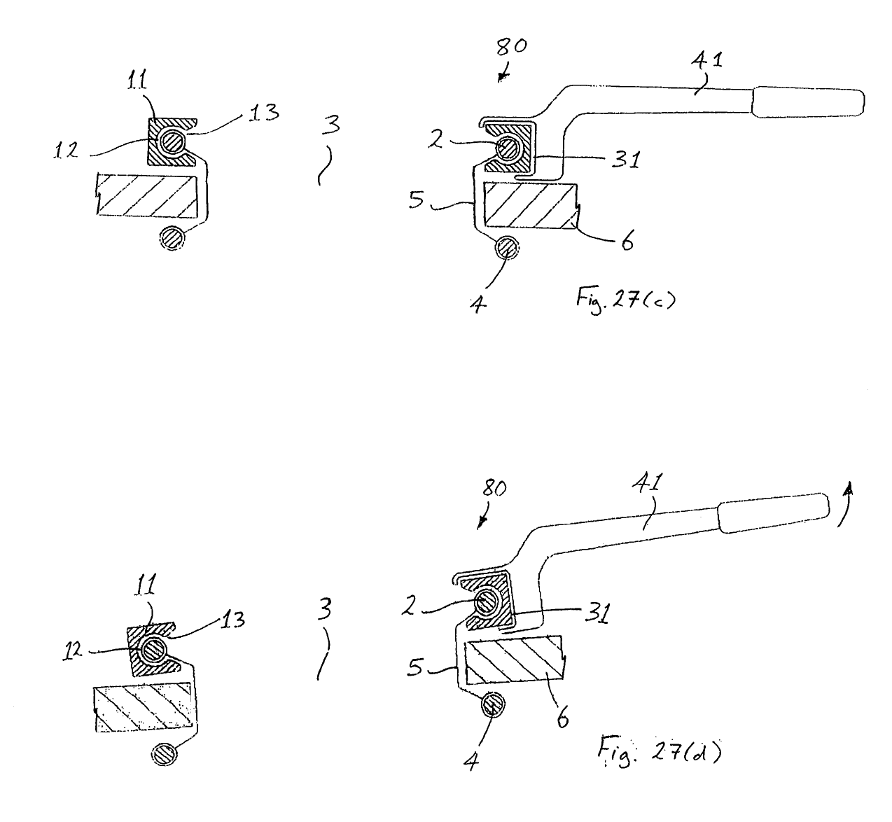

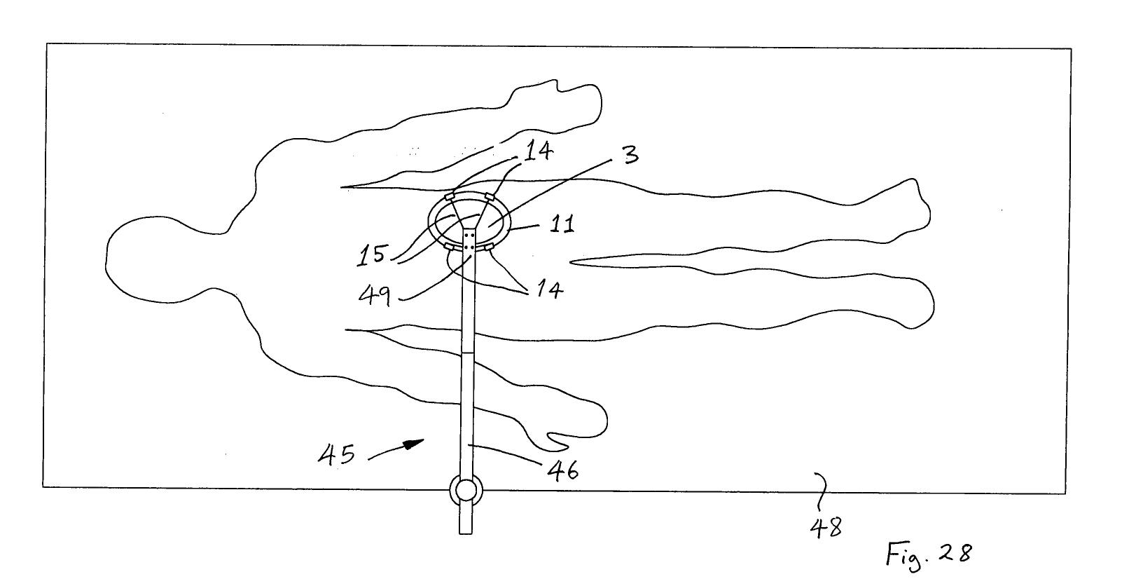

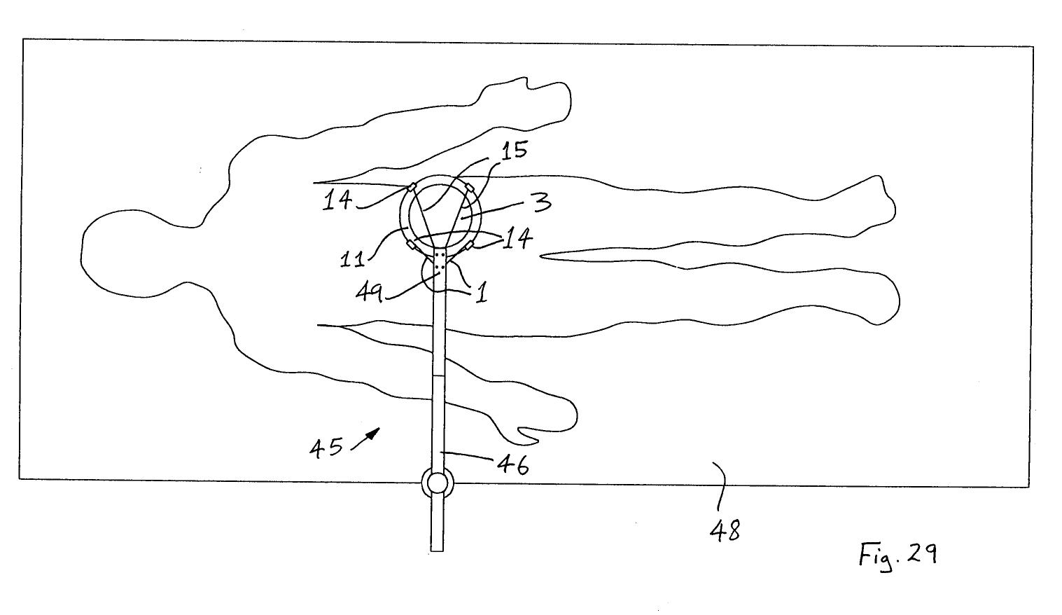

In FIGS. 21 to 25, there is illustrated a further wound retractor device 35 according to the invention, which is similar to the device 30 of FIGS. 18 to 20, and similar elements in FIGS. 21 to 25 are assigned the same reference numerals.

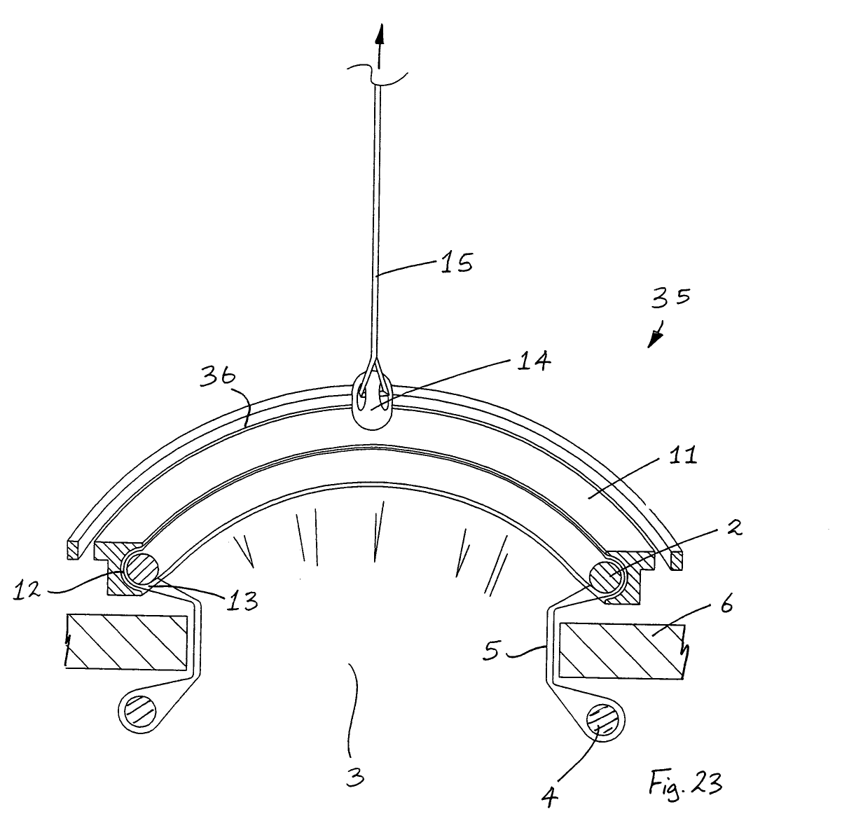

In this case, the engagement ring member 11 comprises a slot 36 extending around part of the circumference of the engagement ring member 11, as illustrated in FIGS. 21 and 22. The hook formation 14 is movably received in the slot 36 to enable the hook formation 14 to be slidably moved along the slot 36.

FIGS. 26 and 27 illustrate another wound retractor device 40 according to the invention, which is similar to the device 30 of FIGS. 18 to 20, and similar elements in FIGS. 26 and 27 are assigned the same reference numerals.

In this case the engagement ring member 11 comprises a handle 41 which provides a means of gripping the engagement ring member 11. The handle 41 extends radially outwardly from the engagement ring member 11 to provide enhanced leverage when manipulating the device 40 into a desired location and/or orientation.

The handle 41 comprises a “C”-shaped female recess 31 into which the engagement ring member 11 is receivable to mount the handle 41 to the engagement ring member 11 in a snap-fit manner.

In FIGS. 27(a) and 27(b), there is illustrated a further wound retractor device 70 according to the invention, which is similar to the device 40 of FIGS. 26 and 27, and similar elements in FIGS. 27(a) and 27(b) are assigned the same reference numerals.

In this case, the wire 15 is attached to the handle 41. Pulling on the wire 15 moves the handle 41, and this manipulates the device 70 into a desired location and/or orientation.

FIGS. 27(c) and 27(d) illustrate another wound retractor device 80 according to the invention, which is similar to the device 40 of FIGS. 26 and 27, and similar elements in FIGS. 27(c) and 27(d) are assigned the same reference numerals.

In this case, the connecting sleeve 5 extends in a single layer from the floppy proximal ring member 2 to the distal ring member 4. The connecting sleeve 5 is fixedly attached to both the proximal ring member 2 and the distal ring member 4.

The rigid engagement ring member 11 provides support to the floppy proximal ring member 2.

Referring to FIGS. 28 to 33 there is illustrated another wound retractor device 45 according to the invention, which is similar to the device 10 of FIGS. 9 to 12, and similar elements in FIGS. 28 to 33 are assigned the same reference numerals.

In this case, the device 45 comprises a support to support the engagement ring member 11 in a manipulated location and/or orientation. The support is provided, in this case, by four wires 14, an arm member 46, and a leg member 47.

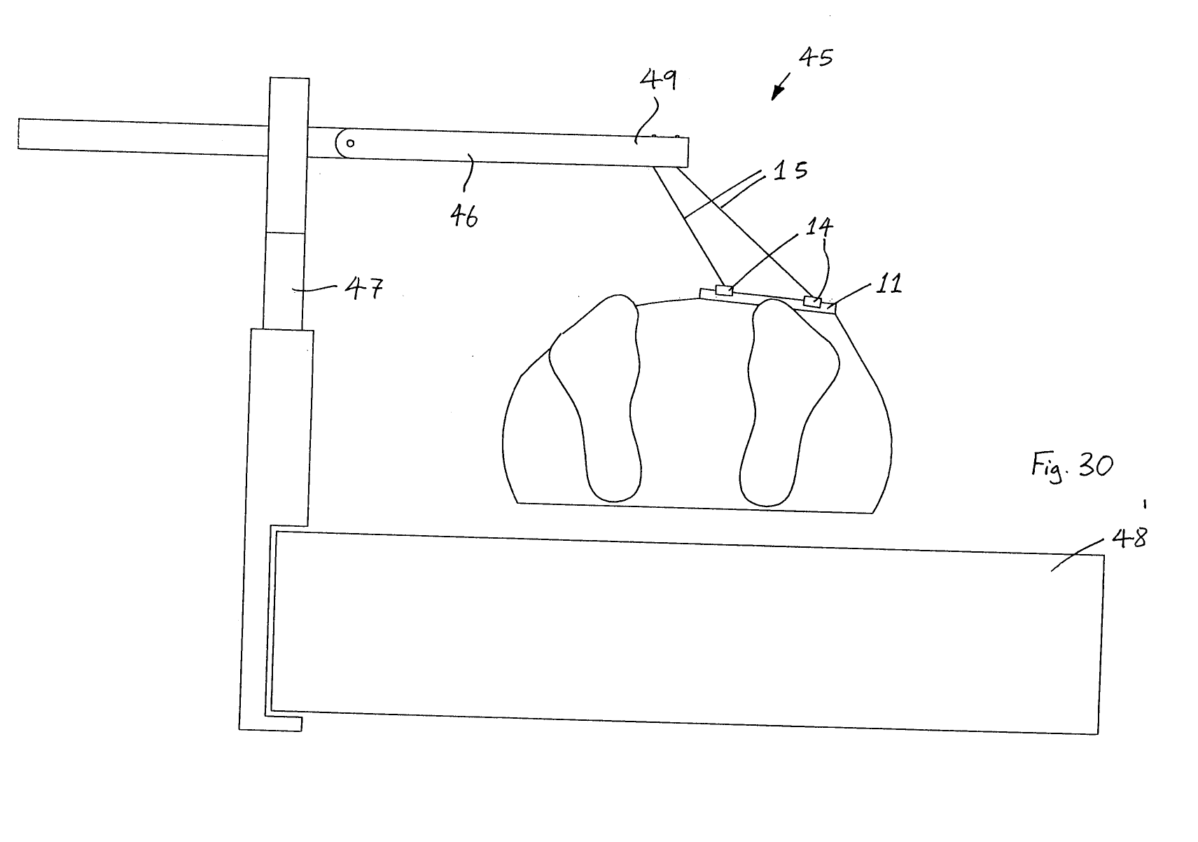

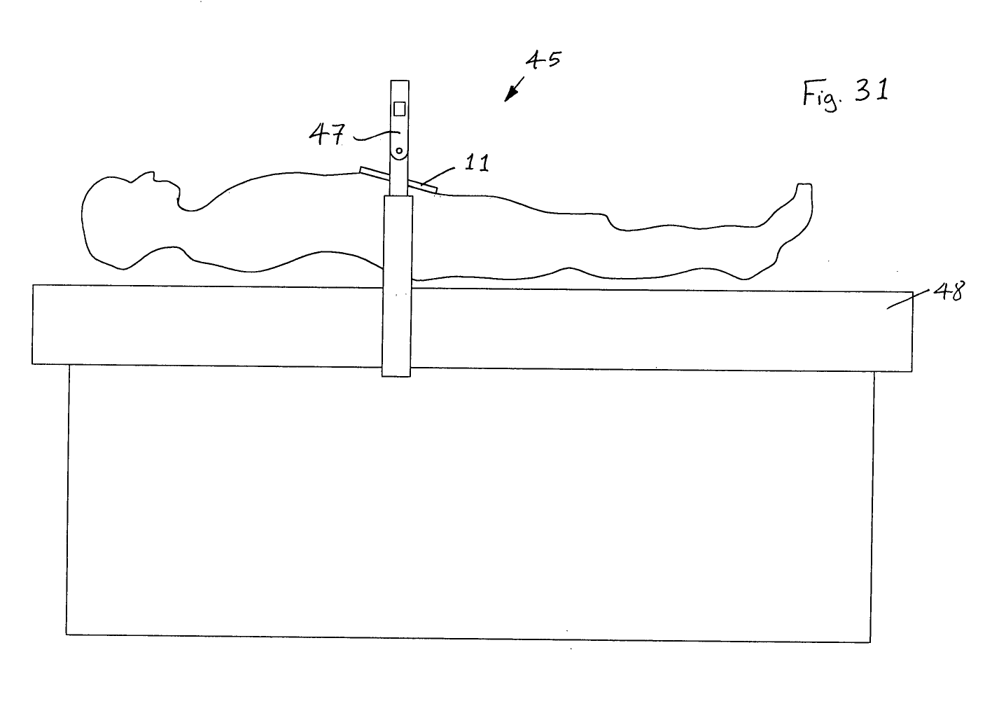

The leg member 47 is releasably attached to a side of a treatment bed 48 a substantial distance from the wound opening 3, extending vertically upwards from the treatment bed 48. The arm member 46, which is supported by the leg member 47, extends from the leg member 47 towards the wound opening 3 with one end 49 positioned adjacent to and substantially above the wound opening 3 to support the engagement ring member 11 from above (FIG. 30).

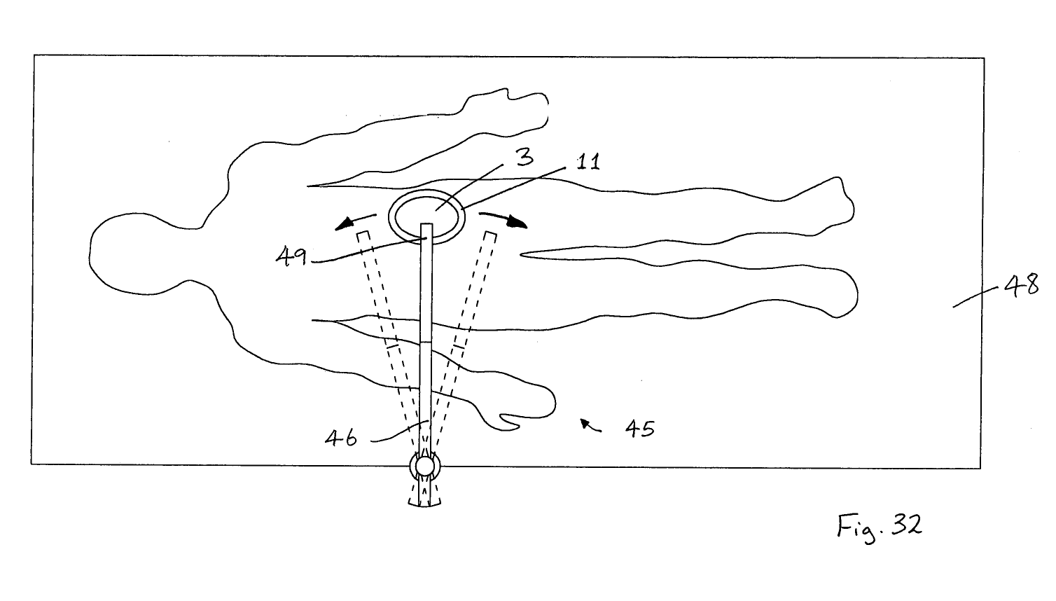

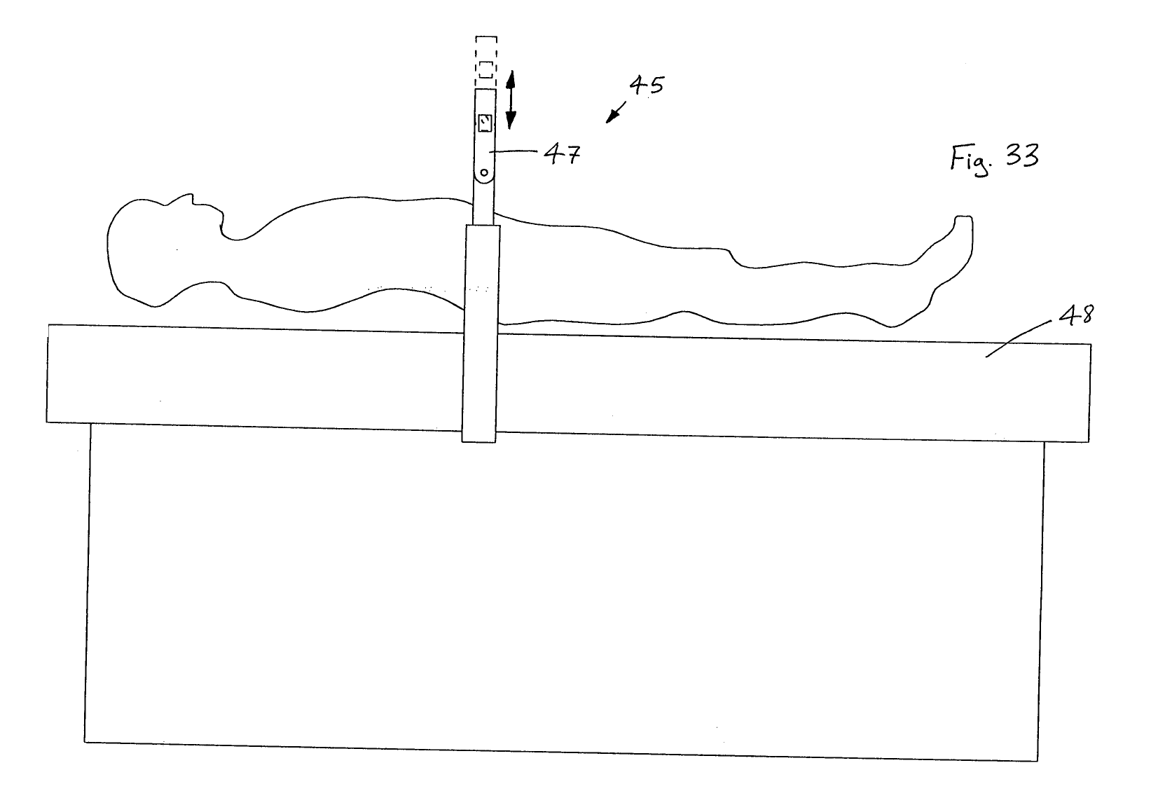

The location and/or orientation of the arm member 46 may be adjusted. For example, by pivoting the arm member 46 relative to the leg member 47 (FIG. 32), or by adjusting the height of the leg member 47 (FIG. 33).

Each wire 15 is looped through a corresponding hook formation 14 on the engagement ring member 11, and is attached to the end 49 of the arm member 46 which is adjacent to the wound opening 3. The wires 15 enable a tensile force to be exerted on the engagement ring member 11 to support the engagement ring member 11 in a manipulated location and/or orientation.

The magnitude and/or direction of the tensile force in the wires 15 may be adjusted to further manipulate the location and/or orientation of the engagement ring member 11. For example, the tensile force exerted may be increased to manipulate the location and/or orientation from a first location and/or orientation to a second location and/or orientation (FIG. 29).

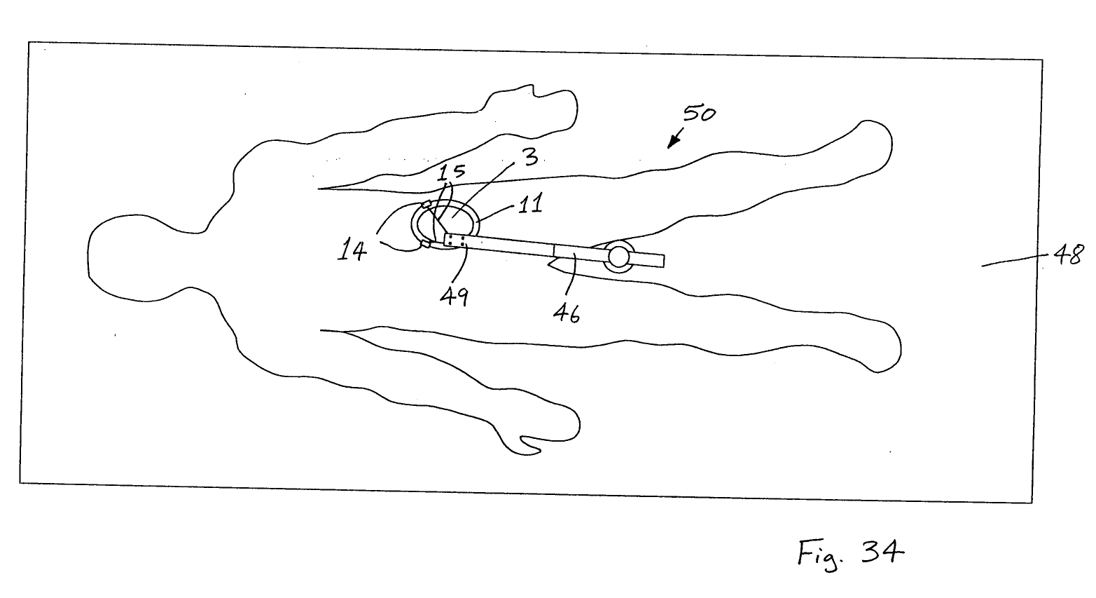

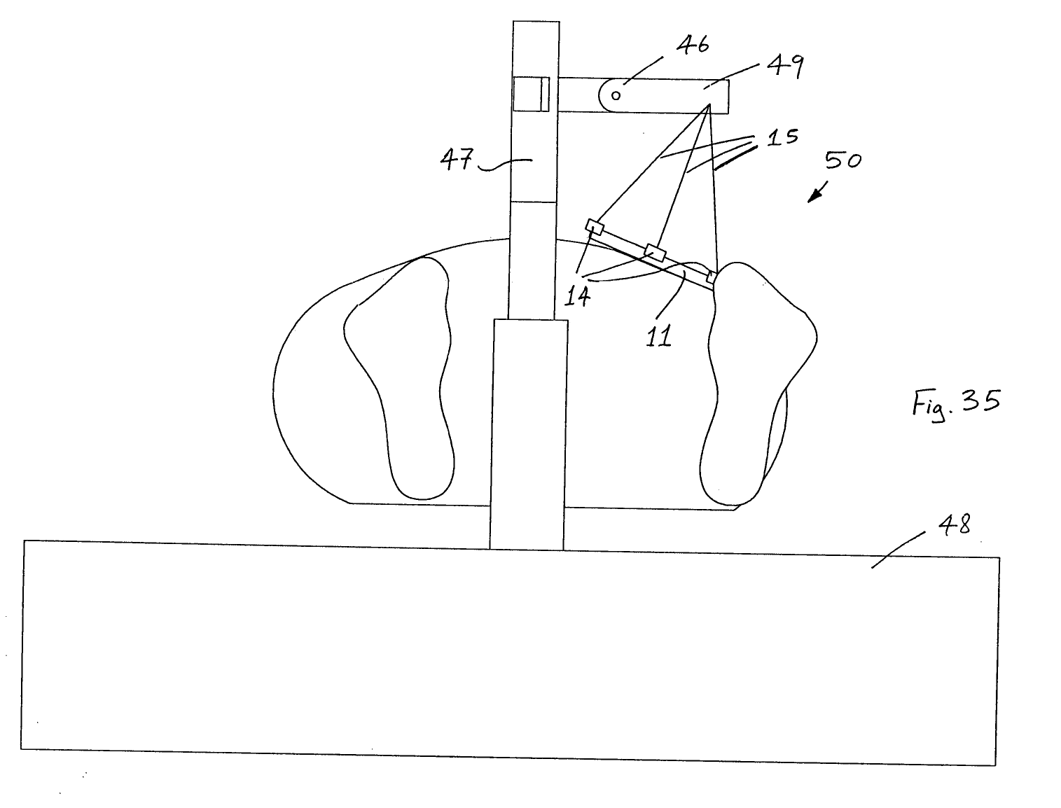

In FIGS. 34 and 35, there is illustrated another wound retractor device 50 according to the invention, which is similar to the device 45 of FIGS. 28 to 33, and similar elements in FIGS. 34 and 35 are assigned the same reference numerals.

In this case, the leg member 47 is releasably attached to the treatment bed 48 extending vertically upwards at a position between the legs of a patient being treated.

The device 50 comprises three wires 15 looped through three corresponding hook formations 14 on the proximal ring member 11.

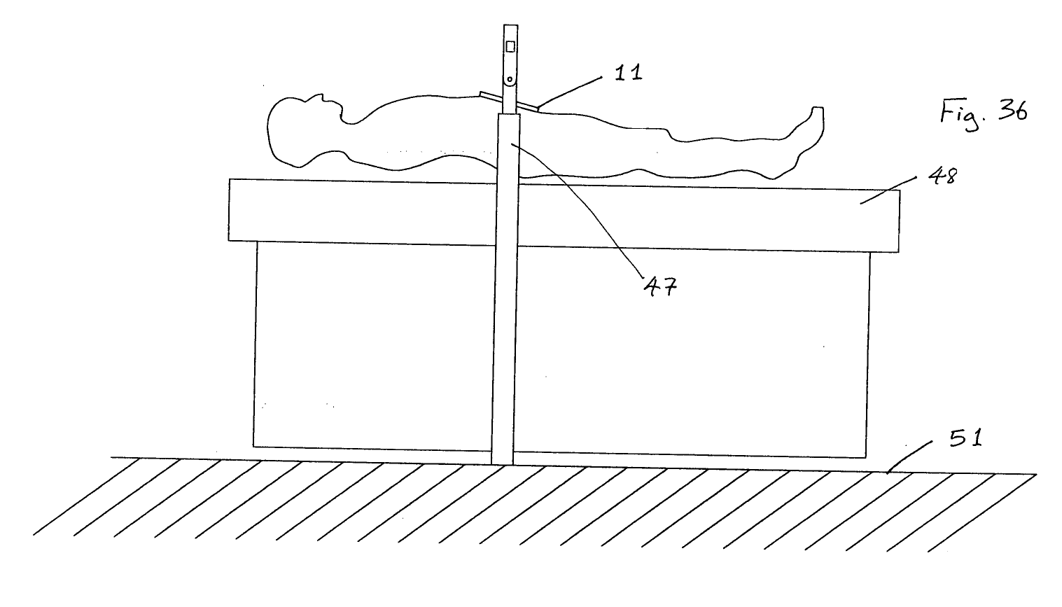

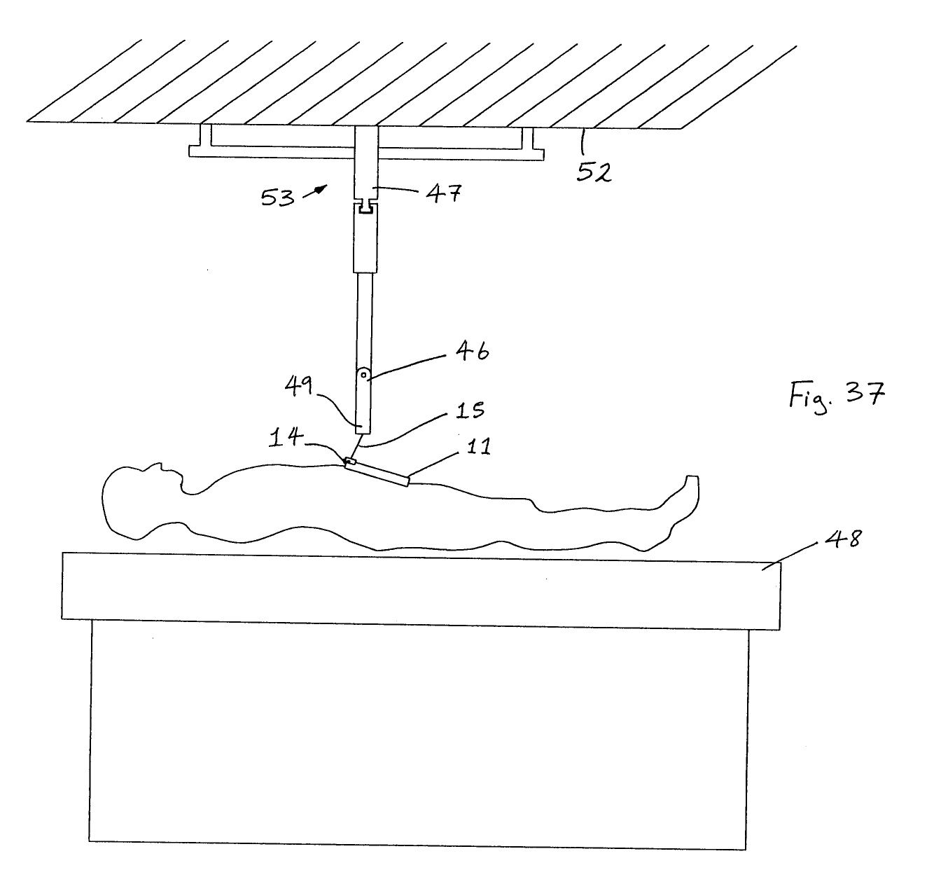

It will be appreciated that the leg member 47 may be releasably attached to any suitable structure, such as the floor 51 (FIG. 36) or the ceiling 52 (FIG. 37). In the case of the leg member 47 being attached to the ceiling 52, a rail structure 53 may be provided to facilitate movement of the arm member 46 in both directions of the horizontal plane of the ceiling 52, as illustrated in FIG. 37.

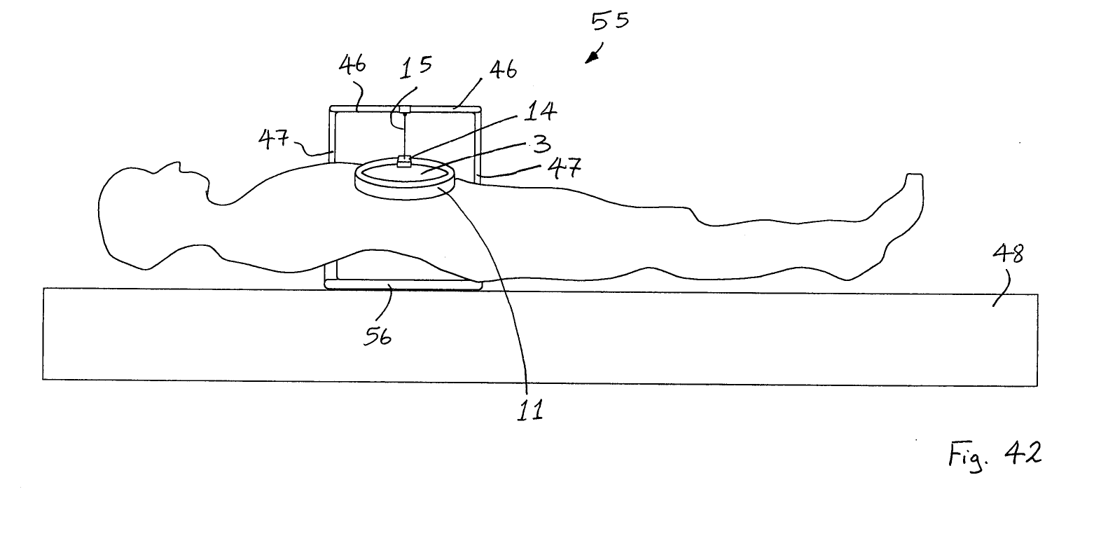

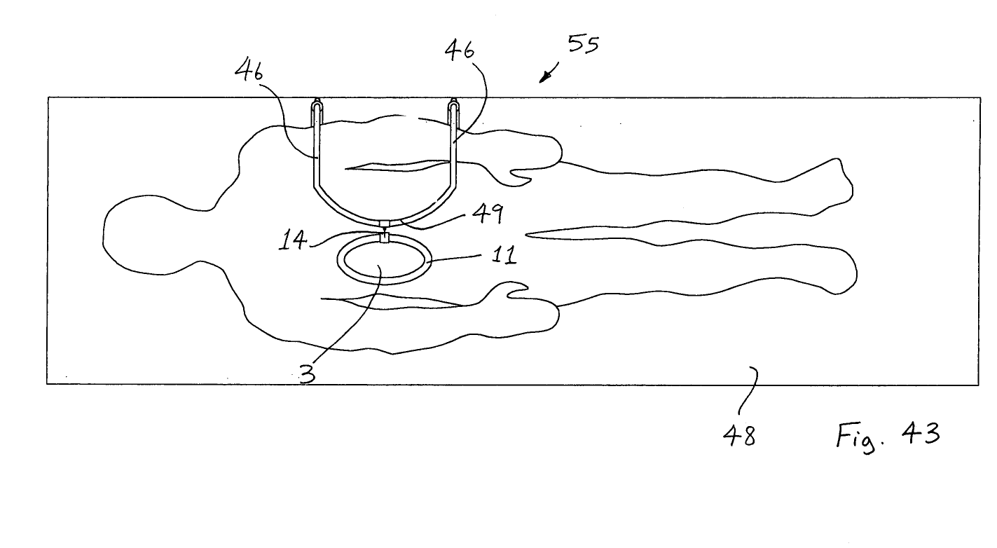

FIGS. 42 to 44 illustrate a further wound retractor device 55 according to the invention, which is similar to the device 45 of FIGS. 28 to 33, and similar elements in FIGS. 42 to 44 are assigned the same reference numerals.

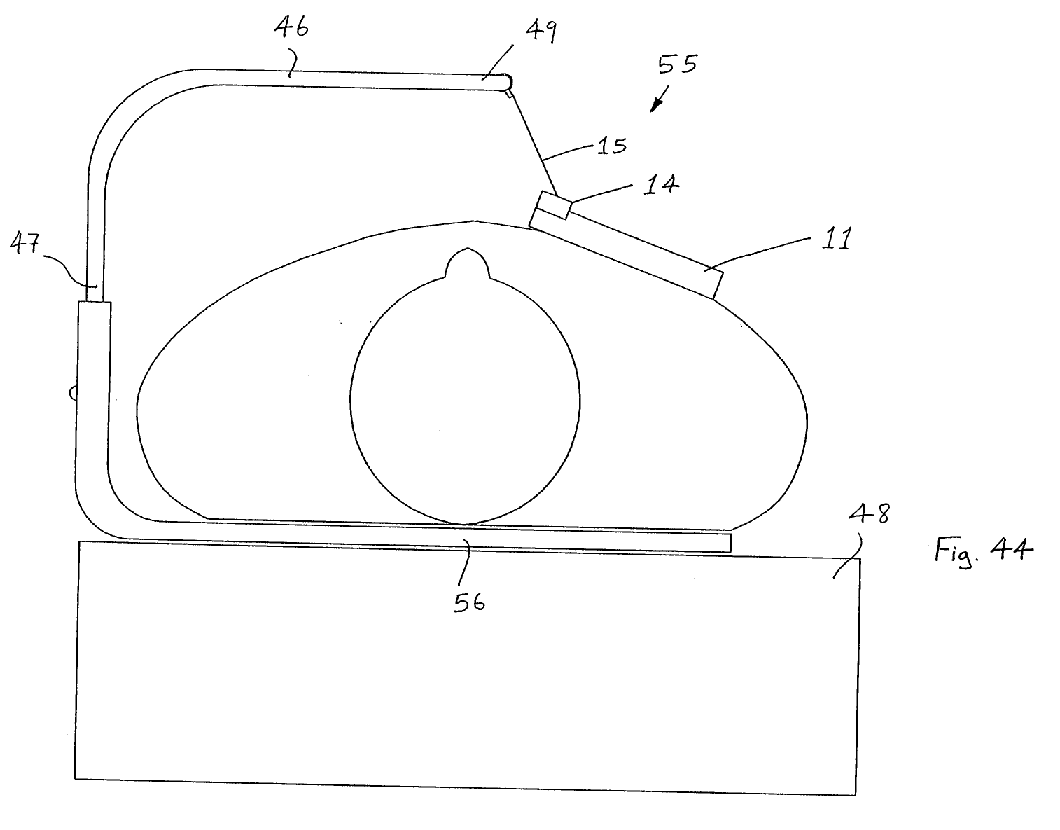

In this case, the device 55 comprises a base member 56 configured for location resting on the treatment bed 48 partially beneath the body of the patient being treated (FIG. 44). Two leg members 47, which are supported by the base member 56, extend vertically upwards from the base member 56. Each leg member 47 supports an arm member 46, with the two arm members 46 meeting at a curved end 49 adjacent the wound opening 3. The wire 15 is looped through the hook formation 14, and is attached to the curved end 49.

Because the device 55 is located partially beneath the patient being treated, this ensures that the device 55 is compact and user-friendly. In particular, the space occupied by the device 55 around the patient being treated is minimised.

In addition, by locating a portion of the base member 56 beneath the body of the patient, this arrangement uses the weight of the patient's body to hold the base member 56 securely in position.

A suitable material for the leg members 47, the arm members 46 and the base member 56 is steel.

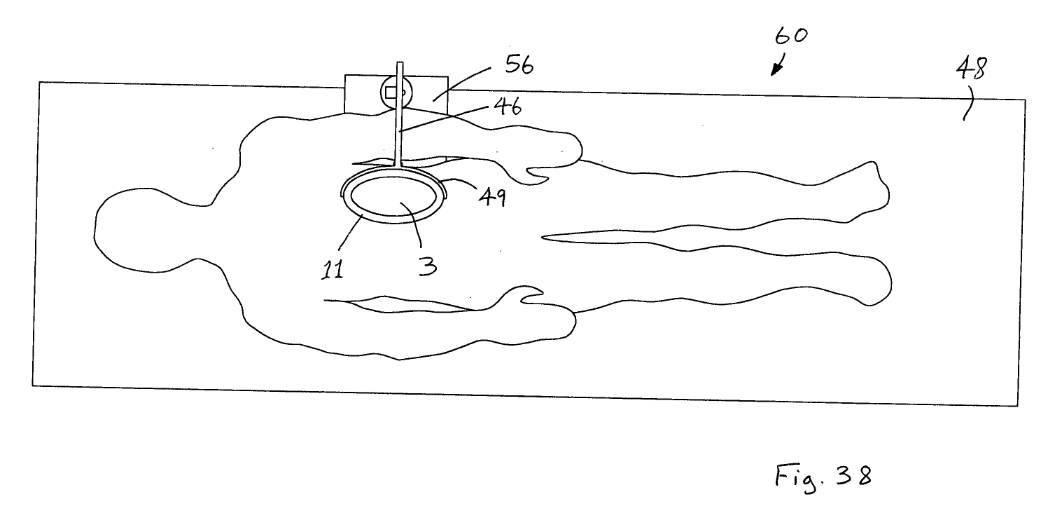

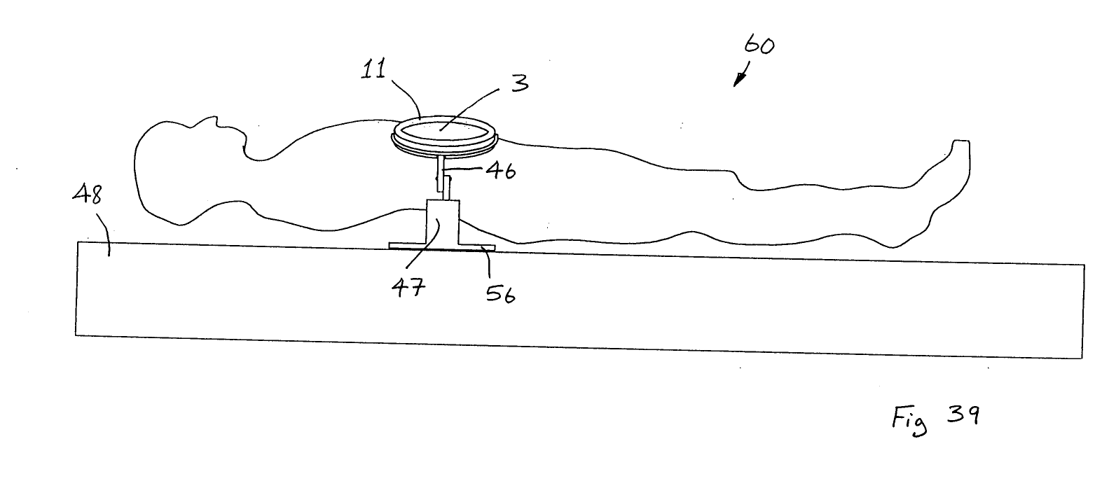

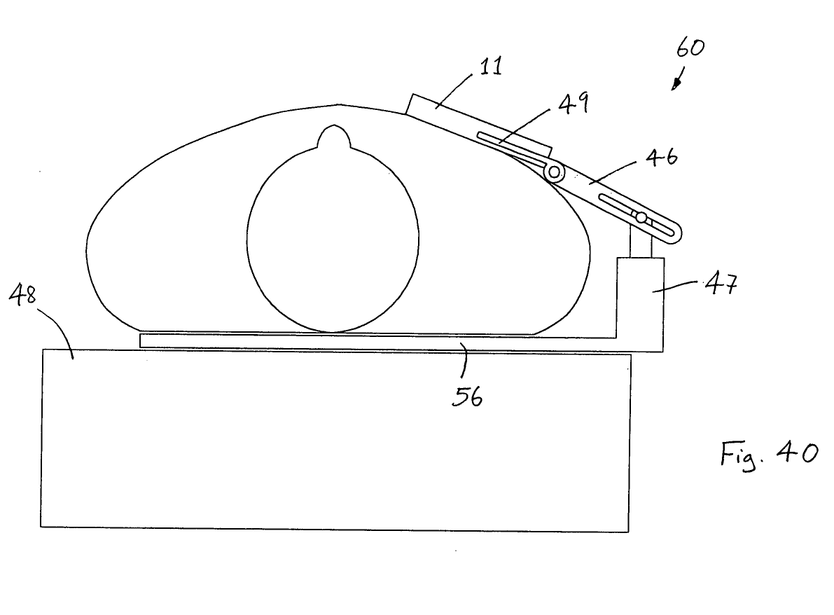

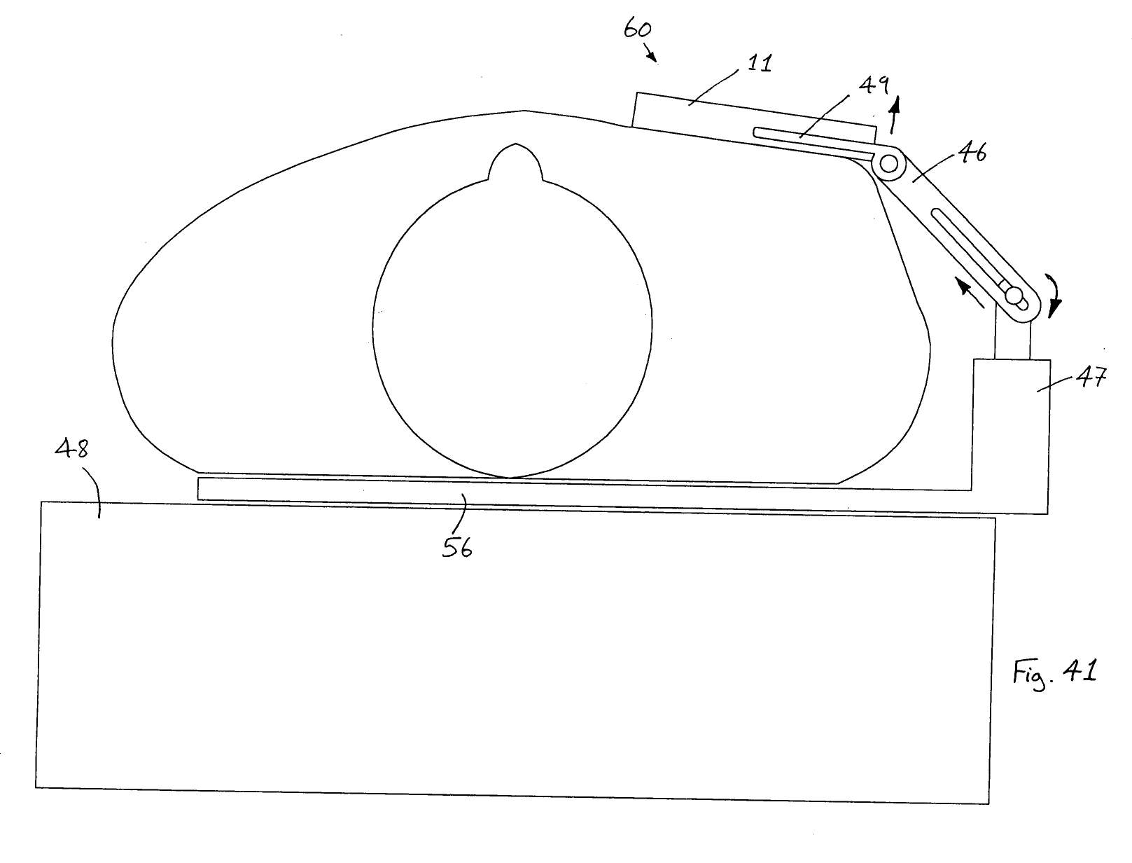

In FIG. 38 to 41 there is illustrated a further wound retractor device 60 according to the invention, which is similar to the device 55 of FIGS. 42 to 44, and similar elements in FIGS. 38 to 41 are assigned the same reference numerals.

In this case, the device 60 comprises one leg member 47 extending vertically upwardly from the base member 56, and one arm member 46 supported by the leg member 47. The arm member 46 is directly attached to the engagement ring member 11 at the end 49.

The location and/or orientation of the arm member 46 is adjustable, as illustrated in FIG. 41, to ensure that the engagement ring member 11 is supported in a desired manipulated location and/or orientation.

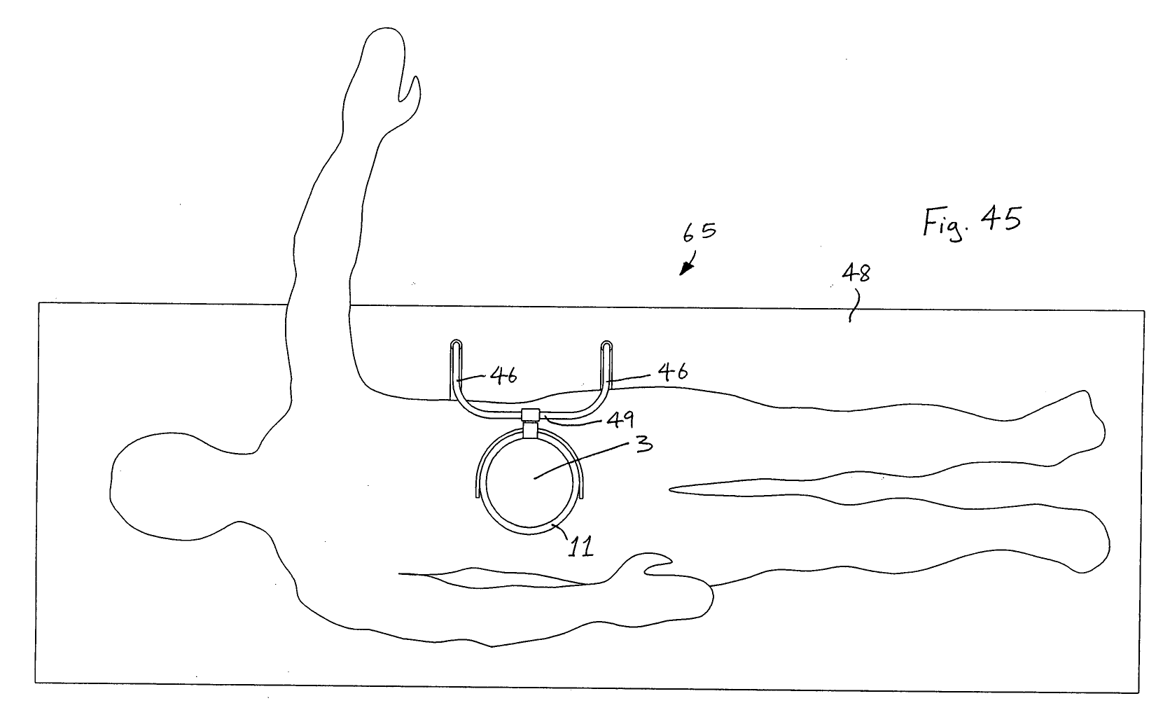

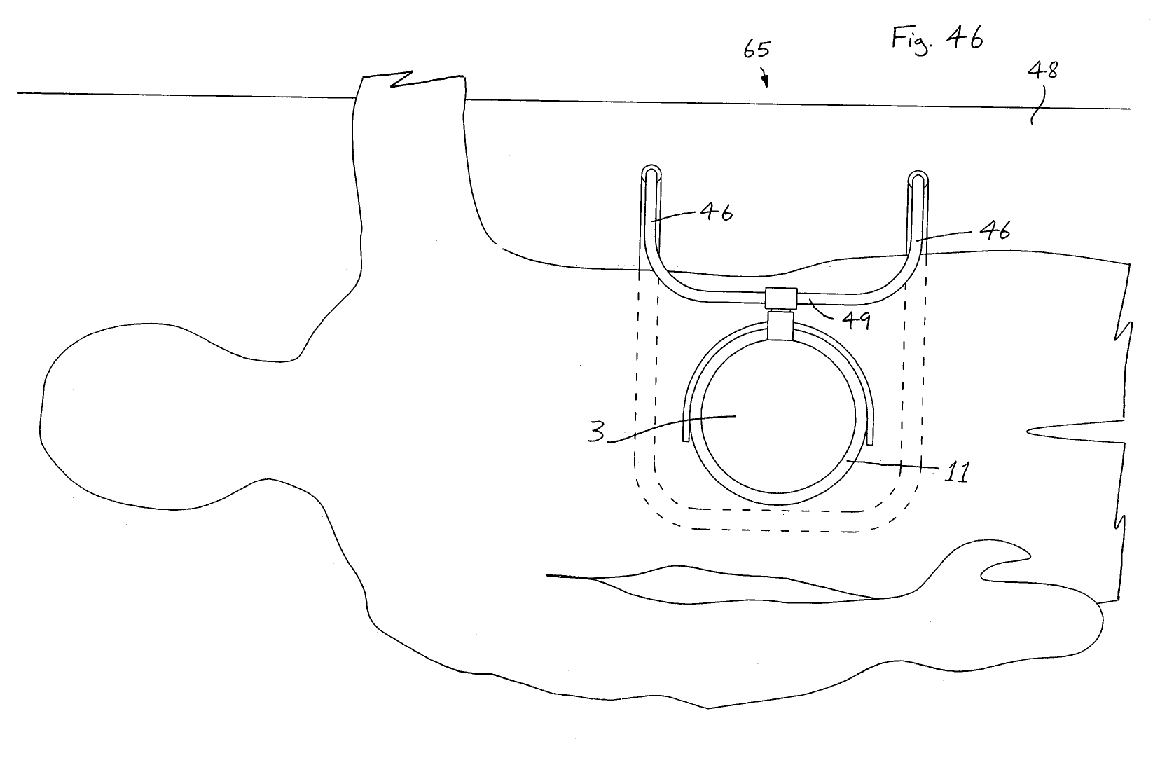

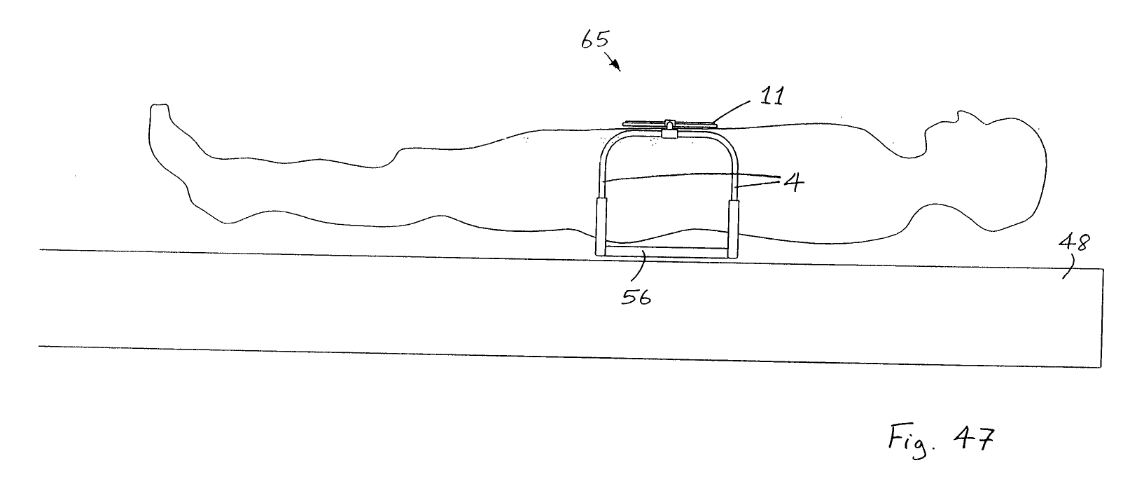

Referring to FIGS. 45 to 47, there is illustrated a further wound retractor device 65 according to the invention, which is similar to the device 55 of FIGS. 42 to 44, and similar elements in FIGS. 45 to 47 are assigned the same reference numerals.

In this case, the two arm members 46 meet at the curved end 49, and the engagement ring member 11 is directly attached to the curved end 49.

It will be appreciated that any suitable means of attachment may be used in addition to or as an alternative to the wires/cables 15 described previously.

It will also be appreciated that such attachment means may be connected to the engagement ring member 11 in any suitable manner in addition to or as an alternative to looping through the hook formation 14. For example a clip could be used.

The invention is not limited to the embodiments hereinbefore described, with reference to the accompanying drawings, which may be varied in construction and detail.

1. A wound retractor device comprising:
a distal member for insertion into a wound opening;
a proximal member for location externally of the wound opening;
a connecting member extending between the distal member and the proximal member to retract laterally the sides of the wound opening; and
an engagable member for facilitating engagement of the wound retractor device and manipulation of the wound retractor device into a desired location and/or orientation.

2. A device as claimed in claim 1 wherein the proximal member comprises the engagable member.

3. A device as claimed in claim 1 wherein the engagable member comprises a separate engagement member for location externally of a wound opening.

4. A device as claimed in claim 3 wherein the engagement member is mountable to the proximal member.

5. A device as claimed in claim 4 wherein the engagement member is mountable to the proximal member in a snap-fit manner.

6. A device as claimed in claim 4 wherein the engagement member comprises a female recess, into which the proximal member is receivable to mount the engagement member to the proximal member.

7. A device as claimed in claim 6 wherein the female recess is substantially “C”-shaped in cross-section.

8. A device as claimed in claim 7 wherein an opening of the female recess faces radially inwardly.

9. A device as claimed in claim 7 wherein an opening of the female recess faces radially outwardly.

10. A device as claimed in claim 7 wherein an opening of the female recess faces distally.

11. A device as claimed in claim 4 wherein the engagement member comprises a lip for engagement with the proximal member to mount the engagement member to the proximal member.

12. A device as claimed in claim 11 wherein the lip protrudes radially outwardly.

13. A device as claimed in claim 12 wherein the engagement member is substantially “L”-shaped in cross-section.

14. A device as claimed in claim 1 wherein the proximal member comprises a ring.

15. A device as claimed in claim 3 wherein the engagement member comprises a ring.

16. A device as claimed in claim 3 wherein the engagement member comprises a grippable element for facilitating gripping of the engagement member.

17. A device as claimed in claim 16 wherein the grippable element is selectively movable relative to a main body portion of the engagement member.

18. A device as claimed in claim 17 wherein the grippable element is slidable relative to the main body portion.

19. A device as claimed in claim 16 wherein the grippable element is mountable to the main body portion.

20. A device as claimed in claim 19 wherein the grippable element is mountable to the main body portion in a snap-fit manner.

21. A device as claimed in claim 19 wherein the grippable element comprises a female recess, into which the main body portion is receivable to mount the grippable element to the main body portion.

22. A device as claimed in claim 21 wherein the female recess is substantially “C”-shaped in cross-section.

23. A device as claimed in claim 16 wherein the main body portion comprises a channel and the grippable element is movably received in the channel.

24. A device as claimed in claim 23 wherein the channel comprises a slot in the main body portion.

25. A device as claimed in claim 23 wherein the channel extends around part of the circumference of the main body portion.

26. A device as claimed in claim 16 wherein the grippable element comprises at least one hook formation.

27. A device as claimed in claim 16 wherein the grippable element comprises a handle.

28. A device as claimed in claim 27 wherein the handle extends radially outwardly from the engagement member.

29. A device as claimed in claims 1 wherein the device comprises a support to support the engagable member in a manipulated location and/or orientation.

30. A device as claimed in claim 29 wherein at least part of the support is configured for positioning substantially above a wound opening to support the engagable member from above.

31. A device as claimed in claim 29 wherein the support comprises a tensioner to exert a tensile force on the engagable member.

32. A device as claimed in claim 31 wherein the magnitude and/or direction of the tensile force is adjustable.

33. A device as claimed in claim 31 wherein the tensioner comprises one or more wires.

34. A device as claimed in claim 29 wherein the support comprises one or more arm members for extending from one or more first locations spaced a substantial distance from a wound opening to one or more second locations adjacent the wound opening.

35. A device as claimed in claim 34 wherein the location and/or orientation of the arm member is adjustable.

36. A device as claimed in claim 34 wherein the support comprises one or more leg members for supporting the one or more arm members in desired locations and/or orientations.

37. A device as claimed in claim 36 wherein the support comprises a base member for supporting the one or more leg members.

38. A device as claimed in claim 37 wherein the base member is configured for location resting on a treatment bed.

39. A device as claimed in claim 38 wherein the base member is configured to at least partially extend beneath the body of a patient being treated.

40. A method for retracting a wound opening, the method comprising the steps of:
providing a wound retractor device comprising a distal member, a proximal member and a connecting member extending between the distal member and the proximal member;
inserting the distal member into the wound opening, and locating the proximal member externally of the wound opening, with the connecting member extending between the distal member and the proximal member to retract laterally the sides of the wound opening; and
engaging the wound retractor device, and manipulating the wound retractor device into a desired location and/or orientation.

41. A method as claimed in claim 40 wherein the proximal member is engaged to manipulate the wound retractor device into a desired location and/or orientation.

42. A method as claimed in claim 40 wherein the wound retractor device comprises a separate engagement member located externally of the wound opening, and the engagement member is engaged to manipulate the wound retractor device into a desired location and/or orientation.

43. A method as claimed in claim 42 wherein the method comprises the step of mounting the engagement member to the proximal member.

44. A method as claimed in claim 42 wherein the method comprises the step of gripping the engagement member at a first region of the engagement member.

45. A method as claimed in claim 44 wherein the method comprises the step of moving the region of gripping of the engagement member from the first region to a second region of the engagement member.

46. A method as claimed in claim 45 wherein the method comprises the step of sliding the region of gripping from the first region to the second region.

47. A method as claimed in claim 40 wherein the method comprises the step of supporting the wound retractor device in a manipulated position and/or orientation.
